(19) United States
(12) Patent Application Publication
(10) Pub. No.: US 2025/0000854 A1
(43) Pub. Date: Jan. 2, 2025

(54) 4-(Aminomethyl)-6-(1-Methyl-1H-Pyrazol-4-YL)Isoquinolin-1(2H)-One Derivatives as MTA-Cooperative Inhibitors of PRMT5

(71) Applicant: BeiGene, Ltd., Grand Cayman (KY)

(72) Inventors: Sanjia XU, Beijing (CN); Jing LI, Beijing (CN); Zhiwei WANG, Beijing (CN)

(73) Assignee: BeiGene, Ltd., Grand Cayman (KY)

(21) Appl. No.: 18/825,776

(22) Filed: Sep. 5, 2024

Related U.S. Application Data
(63) Continuation of application No. PCT/CN2023/081281, filed on Mar. 14, 2023.

(30) Foreign Application Priority Data
Mar. 15, 2022 (WO) PCT/CN2022/080837

(51) Int. Cl. A61K 31/4725; A61P 35/00; C07D 401/04

(52) U.S. Cl. CPC A61K 31/4725; A61P 35/00; C07D 401/04

(57) ABSTRACT

This disclosure provides compounds containing 4-(aminomethyl)-6-(1-methyl-1H-pyrazol-4-yl) isoquinolin-1 (2H)-onestructure, the use thereof for selectively inhibiting the activity of PRMT5 in cooperative with MTA in tumors bearing MTAP^{DEL} mutation, and pharmaceutical compositions comprising the compounds as treatment of various diseases including cancer.

### FIELD OF THE INVENTION

This disclosure provides compounds containing 4-(aminomethyl)-6-(1-methyl-1H-pyrazol-4-yl) isoquinolin-1 (2H)-onestructure, the use thereof for selectively inhibiting the activity of PRMT5 in cooperative with MTA in tumors bearing MTAP^{DEL} mutation, and pharmaceutical compositions comprising the compounds as treatment of various diseases including cancer.

### BACKGROUND OF THE INVENTION

Epigenetic modification is a process that can modify genetic output changing the primary DNA sequence. Epigenetic modification plays an important role in gene expression and regulation, protein production and cell differentiation in multiple dimensions. Typically, this process is reversible and selective, on DNA, its regulatory proteins such as histones and other proteins such as transcription factors [Bradbury, E. M., BioEssays, 1992, 14 (1): pp. 9-16]. PMTs (Protein Methyltransferases) are central players on epigenetic modifications, consisting of two sub-families named PKMTs (Protein Lysine Methyltransferases) and PRMTs (Protein Arginine Methyltransferases) [Copeland, R. A., et al., Oncogene, 2012. 32 (8): pp. 939-46]. PMTs are associated with various human diseases and considered as potential therapeutic targets [Copeland, R. A., et al., Oncogene, 2012, 32 (8): pp. 939-46].

As the name implies, PRMTs catalyze the methylation of the arginine residues of proteins. Besides their primary functions of methylating the histone tails, PRMTs also target on other cellular proteins such as NAB2p, FOXO1, PABP1, Sm D1, etc. [Bedford, M. T., et al., Molecular Cell, 2005, 18 (3): pp. 263-72]. Divided by the products, the 9 mammalian PRMTs can be classified into 3 subtypes: type I (PRMT1, PRMT2, PRMT3, PRMT4, PRMT6 and PRMT8) catalyzes aDMA (asymmetrical dimethylated arginine) formation; type II (PRMT5, PRMT9) catalyzes sDMA (symmetrical dimethylated arginine); and type III (PRMT7) catalyzes MMA (monomethylated arginine) formation [Yang, Y., et al., Nature Reviews Cancer, 2012, 13 (1): pp. 37-50]. In addition, type I/II PRMTs can also catalyze MMA formation as an intermediate to aDMA and sDMA. The PRMTs comprise a pocket to interact with its cofactor SAM (S-adenosyl methionine), and an adjacent pocket to interact with the arginine residue on a protein, namely SAM-pocket and substrate-pocket. The methylation process involves an S_{N}2-like mechanism of transferring an activated methyl group from cofactor SAM to the guanidino group on the arginine residue. [Bedford, M. T., et al., Molecular Cell, 2005, 18 (3): pp. 263-72]. The side product of the process is SAH (S-adenosyl-L-homocysteine).

The overall arginine level in cells is roughly 1500:3:2:1 for Arg: aDMA: MMA: sDMA, and PRMT5 accounts for the vast majority of sDMA formation [Dhar, S., et al., Scientific Reports, 2013, 3: 1311]. In contrast with PRMT1, the major type I PRMT which functions on its own in cells, PRMT5 binds to MEP50 (Methylosome Protein 50) to form a heterocomplex that is often elevated in cancer cells and correlates to poor patient survival [Gao, G., et al., Nucleic Acids Research, 2019, 47 (10): pp. 5038-48]. PRMT5 promotes tumerigenesis in varied mechanisms. PRMT5 is a strong repressor of numerous genes; when PRMT5 methylates histones H₂a and H₄ on Arg3 and histone H₃ on Arg8, it represses gene transcripts that involved in differentiation, transformation, cell-cycle progression and tumor suppression [Karkhanis, V., et al., Trends in Biochemical Sciences, 2011, 36 (12): pp. 633-41]. Besides its epigenetic roles, PRMT5 may also regulates RNA-binding proteins such as splicing factors. For instance, a reproducible event was observed in PRMT5 knockout mice, in which exon 6 skipping of MDM4 (Murine Double Minute 4) occurred and p53 was released to upregulate p53 pathway [Gerhart, S. V., et al., Scientific Reports, 2018, 8:9711]. In addition, PRMT5 could directly influence key proliferation pathways by direct methylation of p53 [Jansson, M., et al., Nature Cell Biology, 2008, 10 (12): pp. 1431-9], EGFR [Hsu, J.-M., et al., Nature Cell Biology, 2011, 13 (2): pp. 174-81], PI3K [Wei, T.-Y. W., et al., Cellular Signaling, 2014, 26 (12): pp. 2940-50], etc. Thus, PRMT5 has a good potential to become a clinically relevant target.

On the other hand, PRMT5 is an essential gene in normal tissues, and the systemic inhibition of PRMT5 may result in significant liabilities, especially hematologic toxicity [Ahnert. J. R., et al., Journal of Clinical Oncology, 2021, 39 (15-suppl): p. 3019]. Therefore, strategies to selectively block the PRMT5 activities in tumor cells are required for a safer therapy.

Homozygous deletion of tumor depressor CDKN2A (Cyclin Dependent Kinase Inhibitor 2A) occurs in about 15% of all tumor types. Interestingly, the mutation frequently involves the co-deletion of proximate genes existing in 9p21, including the gene that encodes MTAP (Methylthioadenosine Phosphorylase) [Firestone, R. S., et al., Journal of American Chemical Society, 2017, 139 (39): p. 13754-60]. As a result of MTAP deletion, MTA (methylthioadenosine), the substrate of MTAP, accumulates. MTA is structurally related to SAM, and is a weak ligand/inhibitor of PRMT5 that occupies the same pocket with SAM. The formation of MTA-PRMT5 complex provides chances for further PRMT5 inhibition by formation of a tertiary complex. In such way, a correlation of MTAP null status and dependency of PRMT5 is established through MTA concentration level, to provide a precise oncological therapy.

Currently, most of the clinical-stage PRMT5 inhibitors are unable to differentiate normal cells and cancer cells, based on a SAM/MTA competitive mechanism (JNJ64619178, PF06939999, PRT543, and PRT811) or a non-MTA cooperative mechanism (GSK3326595). There thus remain unmet and continuous medical needs for potent and selective MTA-cooperative PRMT5 inhibitors.

### SUMMARY OF THE INVENTION

One objective of the present invention is to provide compounds and derivatives which function to act as PRMT5 inhibitors, and methods of preparation and uses thereof.

Aspect 1. A compound of Formula (I):

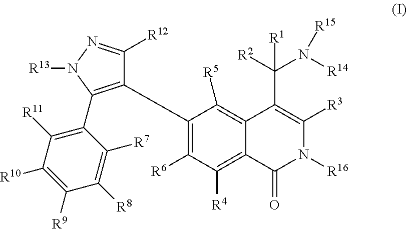

or a N-oxide thereof, or a pharmaceutically acceptable salt thereof, or a stereoisomer thereof, or a deuterated analog thereof, wherein:
R¹, R², R³, R⁴, R⁵, R⁶, R⁷, R⁸, R¹¹ and R¹² are each independently selected from hydrogen, halogen, —C₁₋₈alkyl, —C₃-C₈cycloalkyl, —CN, —OR^{1a}, —NR^{1a}R^{1b}, —COR^{1a}, —CO₂R^{1a}, —CONR^{1a}R^{1b} or —NR^{1a}COR^{1b}, wherein each of —C₁₋₈alkyl and —C₃-C₈cycloalkyl is optionally substituted with at least one substituent selected from halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}OR^{1d}, —NR^{1c}CO₂R^{1d}, or —NR^{1c}SO₂R^{1d};
R^{1a} and R^{1b} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁹ and R¹⁰ are each independently selected from hydrogen, halogen, —C₁₋₈alkyl, C₃-C₈cycloalkyl, —CN, —OR^{9a}, —NR^{9a}R^{9b} or —NR^{9a}COR^{9b}, wherein each of —C₁₋₈alkyl and C₃-C₈cycloalkyl is optionally substituted with at least one substituent R^{9d}; or
R⁹ and R¹⁰ together with the carbon atoms to which they are attached, form a 5-6 membered saturated or partially or completely unsaturated (preferably completely unsaturated, i.e., aromatic) ring, said ring comprising 0-3 heteroatoms independently selected from nitrogen, oxygen or sulfur; said ring is optionally substituted with at least one substituent R^{9e};
R^{9e}, at each occurrence, is independently hydrogen, halogen, —C₁₋₈alkyl, —C₂₋₈alkenyl, —C₂₋₈alkynyl, —C₁₋₈alkoxy, —C₃-C₈cycloalkyl, oxo, 3- to 8-membered heterocyclyl, C₆-C₁₂aryl, 5- to 12-membered heteroaryl, —CN, —SO₂R⁹³, —SO₂NR^{9a}R^{9b}, —COR^{9a}, —CO₂R^{9a}, —CONR^{9a}R^{9b}, —NR^{9a}R^{9b}, —NR^{9a}COR^{9b}, —NR^{9a}CO₂R^{9b} or —NR^{9a}SO₂R^{9b}, wherein each of —C₁₋₈alkyl, —C₂₋₈alkenyl, —C₂₋₈alkynyl, —C₁₋₈alkoxy, C₃-C₈cycloalkyl, 3- to 8-membered heterocyclyl, C₆-C₁₂aryl or 5- to 12-membered heteroaryl is optionally substituted with at least one substituent R^{9d}.
R^{9a} and R^{9b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R^{9f}.
R^{9d} and R^{9f}, at each occurrence, are each independently halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R¹³, R¹⁴, R¹⁵ and R¹⁶ are each independently selected from hydrogen, —C₁₋₈alkyl, —C₃-C₈cycloalkyl or —C₆-C₁₂aryl, wherein each of —C₁₋₈alkyl, —C₃-C₈cycloalkyl and —C₆-C₁₂aryl is optionally substituted with at least one substituent selected from hydrogen, halogen, —C₁₋₈alkyl, —C₂₋₈alkenyl, —C₂₋₈alkynyl, —NR^{13a}R^{13b}, —OR^{13a}, oxo, —C₃-C₈cycloalkyl, 3- to 8-membered heterocyclyl, —C₆-C₁₂aryl, 5- to 12-membered heteroaryl, or —CN;
R^{13a} and R^{13b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, wherein each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{13c},
R^{13c} is independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl or —CN, wherein each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl is optionally substituted with at least one hydrogen, halogen, hydroxy, —C₁₋₈alkyl, —C₁₋₈alkoxy, —CN, —NH₂ or oxo.

Aspect 2. The compound of Aspect 1, wherein the compound is selected from formula (IIa) or (IIb)

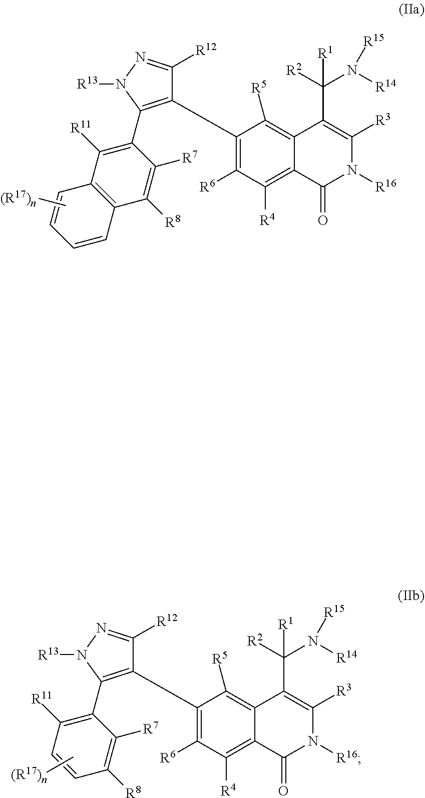

wherein R¹, R², R³, R⁴, R⁵, R⁶, R⁷, R⁸, R¹, R¹², R¹³, R¹⁴, R¹⁵ and R¹⁶ are each as defined as Aspect 1;
at each of its occurrences, R¹⁷ is independently selected from hydrogen, halogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —SO₂R^{17a}, —SO₂NR^{17a}R^{17b}, —COR^{17a}, —CO₂R^{17a}, —CONR^{17a}R^{17b}, —OR^{17a}, —NR^{17a}R^{17b}, —NR^{17a}COR^{17b}, —NR^{17a}CO₂R^{17b}, or —NR^{17a}SO₂R^{17b}, wherein each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{17c}, —SO₂R^{17c}, —SO₂NR^{17c}R^{17d}, —COR^{17c}, —CO₂R^{17c}, —CONR^{17c}R^{17d}, —NR^{17c}R^{17d}, —NR^{17c}COR^{17d}, —NR^{17c}CO₂R^{17d}, or —NR^{17c}SO₂R^{17d}.
R^{17a} and R^{17b} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{17c} and R^{17d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
n is 0, 1, 2, 3 or 4;
m is 0, 1 or 2.

Aspect 3. The compound of Aspect 1 or Aspect 2, wherein the compound is selected from formula (IIIa) or (IIIb)

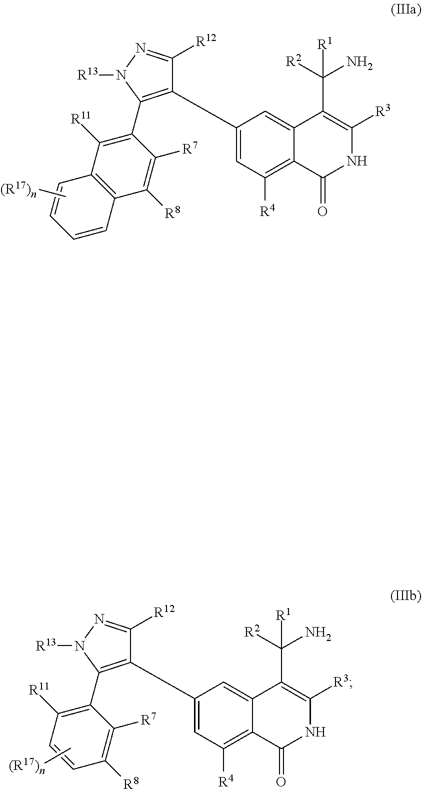

wherein R¹, R², R³, R⁴, R⁵, R⁶, R⁷, R⁸, R¹¹, R¹², R¹³, R¹⁴, R¹⁵ and R¹⁶ are each as defined as Aspect 1; is defined as Aspect 2;
R¹⁷, n and m are each defined as Aspect 2.

Aspect 4. The compound of any one of the preceding Aspects, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b}, or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl and cycloheptyl, cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR¹⁸, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}OR^{1d}, —NR^{1c}O₂R^{1d}, or —NR^{1c}SO₂R^{1d};
R^{1a}, R^{1b}, R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent selected from halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 5. The compound of any one of the preceding Aspects, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b}, or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 6. The compound of any one of the preceding Aspects, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl or cyclooctyl; preferably, R¹, R², R³, R⁴, R⁵, R⁶ and R¹² are each independently selected from hydrogen, methyl, ethyl, propyl, butyl, pentyl, hexyl, heptyl, octyl; more preferably, R¹, R², R³, R⁴, R⁵, R⁶ and R¹² are each independently selected from hydrogen.

Aspect 7. The compound of any one of the preceding Aspects, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b}, or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 8. The compound of any one of the preceding Aspects, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR₁ₐR^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 9. The compound of any one of the preceding Aspects, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl or cyclooctyl; preferably, R⁷ is each independently selected from hydrogen, —F, —Cl, —Br, —I, methyl, ethyl, propyl, butyl, pentyl, hexyl, heptyl, octyl; more preferably, R⁷ is independently selected from hydrogen, —F, —Cl, —Br or —I.

Aspect 10. The compound of any one of the preceding Aspects, wherein R⁸ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b} or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}OR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 11. The compound of any one of the preceding Aspects, wherein R⁸ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 12. The compound of any one of the preceding Aspects, wherein R⁸ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl or cyclooctyl; preferably, R⁸ is each independently selected from hydrogen, —F, —Cl, —Br, —I, —CH₂F, —CHF₂, —CF₃, —C₂F₅, methyl, ethyl, propyl, butyl, pentyl, hexyl, heptyl, octyl; more preferably, R⁸ is independently selected from hydrogen, —CH₂F, —CHF₂, —CF₃, —C₂F₅, —F, —Cl, —Br or —I.

Aspect 13. The compound of any one of the preceding Aspects, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{9a}, —NR^{9a}R^{9b} or —NR^{9a}COR^{9b}, wherein each of methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with at least one substituent R^{9d};
R^{9a} and R^{9b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9f}.
R^{9d} and R^{9f}, at each occurrence, are each independently —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 14. The compound of any one of the preceding Aspects, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy, each of said methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 15. The compound of any one of the preceding Aspects, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy.

Aspect 16. The compound of anyone of Aspects 1-12, wherein R⁹ and R¹⁰ together with the carbon atoms to which they are attached, form a 5 or 6 membered saturated or partially or completely unsaturated (preferably completely unsaturated, i.e., aromatic) ring, said ring comprising 0, 1, 2 or 3 heteroatoms independently selected from nitrogen, oxygen or sulfur; said ring is optionally substituted with at least one substituent R^{9e};
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN, —SO₂R^{9a}, —SO₂NR^{9a}R^{9b}, —COR⁹⁴, —CO₂R^{9a}, —CONR^{9a}R^{9b}, —NR^{9a}R^{9b}, —NR^{9a}COR^{9b}, —NR^{9a}CO₂R^{9b} or —NR^{9a}SO₂R^{9b}, wherein each of methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl or 5- to 12-membered heteroaryl is optionally substituted with at least one substituent R^{9d};
R^{9a} and R^{9b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9f};
R^{9d} and R^{9f}, at each occurrence, are each independently —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 17. The compound of anyone of Aspects 1-12 or 16, wherein R⁹ and R¹⁰ together with the carbon atoms to which they are attached, form a 5 or 6 membered aromatic ring, said ring comprising 0, 1, 2 or 3 heteroatoms independently selected from nitrogen, oxygen or sulfur; said ring is optionally substituted with at least one substituent R^{9e};
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN, wherein each of methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl or 5- to 12-membered heteroaryl is optionally substituted with at least one substituent —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 18. The compound of anyone of Aspects 1-12 or 16-17, wherein R⁹ and R¹⁰ together with the carbon atoms to which they are attached, form a phenyl ring; said ring is optionally substituted with at least one substituent R⁹;
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN.

Aspect 19. The compound of any one of the preceding Aspects, wherein R¹¹ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b} or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR¹⁴, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 20. The compound of any one of the preceding Aspects, wherein R¹ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 21. The compound of any one of the preceding Aspects, wherein R¹¹ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl or —CN; preferably, R¹¹ is each independently selected from hydrogen, —F, —Cl, —Br, —I, —CH₂F, —CHF₂, —CF₃, —C₂F₅, methyl, ethyl, propyl, butyl, pentyl, hexyl, heptyl, octyl or —CN; more preferably, R¹¹ is —CN.

Aspect 22. The compound of any one of the preceding Aspects, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl, wherein each of methyl, ethyl, propyl, butyl, pentyl, hexyl, heptyl, octyl, cyclopropyl, cyclobutyl, cyclopentyl, cyclohexyl, cycloheptyl, cyclooctyl and phenyl is optionally substituted with at least one substituent elected from hydrogen, —F, —Cl, —Br, —I, methyl, ethyl, propyl, butyl, pentyl, hexyl, heptyl, octyl, —C₂₋₈alkenyl, —C₂₋₈alkynyl, —NR^{13a}R^{13b}, —OR^{13a}, oxo, cyclopropyl, cyclobutyl, cyclopentyl, cyclohexyl, cycloheptyl, cyclooctyl, 3- to 8-membered heterocyclyl, phenyl, 5- to 12-membered heteroaryl, or —CN;
R^{13a} and R^{13b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{13c};
R^{13c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN, wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one hydrogen, —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —CN, —NH₂ or oxo.

Aspect 23. The compound of any one of the preceding Aspects, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl, wherein each of methyl, ethyl, propyl, butyl, pentyl, hexyl, heptyl, octyl, cyclopropyl, cyclobutyl, cyclopentyl, cyclohexyl, cycloheptyl, cyclooctyl and phenyl is optionally substituted with at least one substituent elected from hydrogen, —F, —Cl, —Br, —I, methyl, ethyl, propyl, butyl, pentyl, hexyl, heptyl, octyl, —C₂₋₈alkenyl, —C₂₋₈alkynyl, —NR^{13a}R^{13b}, —OR^{13a}, oxo, cyclopropyl, cyclobutyl, cyclopentyl, cyclohexyl, cycloheptyl, cyclooctyl, 3- to 8-membered heterocyclyl, phenyl, 5- to 12-membered heteroaryl, or —CN;
R^{13a} and R^{13b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 24. The compound of any one of the preceding Aspects, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl; preferably R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl; more preferably R¹³, R¹⁴, R¹⁵, R¹⁶ are each independently selected from hydrogen, methyl, ethyl, propyl, butyl, pentyl, hexyl, heptyl, octyl.

Aspect 25. The compound of any one of the preceding Aspects, wherein R¹³, R¹⁴, R¹⁵, R¹⁶ are each independently selected from hydrogen, methyl; preferably R¹³ is methyl and R¹⁴, R¹⁵ and R¹⁶ are each hydrogen.

Aspect 26. The compound of any one of the preceding Aspects, wherein R¹⁷ is independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —SO₂R^{17a}, —SO₂NR^{17a}R^{17b}, —COR^{17a}, —CO₂R^{17a}, —CONR^{17a}R^{17b}, —OR^{17a}, —NR^{17a}R^{17b}, —NR^{17a}COR^{17b}, —NR^{17a}CO₂R^{17b}, or —NR^{17a}SO₂R^{17b}, wherein each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{17c}, —SO₂R^{17c}, —SO₂NR^{17c}R^{17d}, —COR^{17c}, —CO₂R^{17c}, —CONR^{17c}R^{17d}, —NR^{17c}R^{17d}, —NR^{17c}COR^{17d}, —NR^{17c}CO₂R^{17d}, or —NR^{17c}SO₂R^{17d};
R^{17c} and R^{17b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{17c} and R^{17d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 27. The compound of any one of the preceding Aspects, wherein R¹⁷ is independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —SO₂R^{17a}, —SO₂NR^{17a}R^{17b}, —COR^{17a}, —CO₂R^{17a}, —CONR^{17a}R^{17b}, —OR^{17d}, —NR^{17a}R^{17b}, —NR^{17a}COR^{17b}, —NR^{17a}CO₂R^{17b}, or —NR^{17a}SO₂R^{17b};
R^{17a} and R^{17b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 28. The compound of any one of the preceding Aspects, wherein R¹⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, —CN, cyclopropoxy, cyclobutoxy, cyclopentoxy, cyclohexoxy, cyclohepthoxy, cyclooctoxy.

Aspect 29. The compound of any one of the preceding Aspects, wherein the

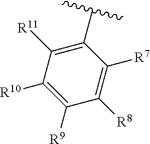

moiety is

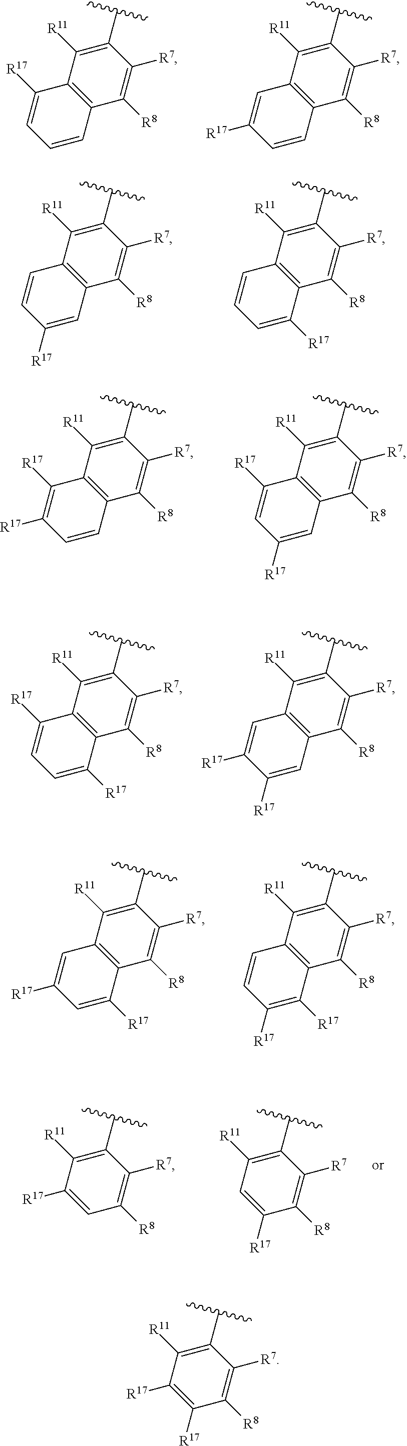

Aspect 30. The compound of any one of the preceding Aspects, wherein the

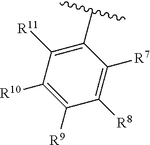

moiety is

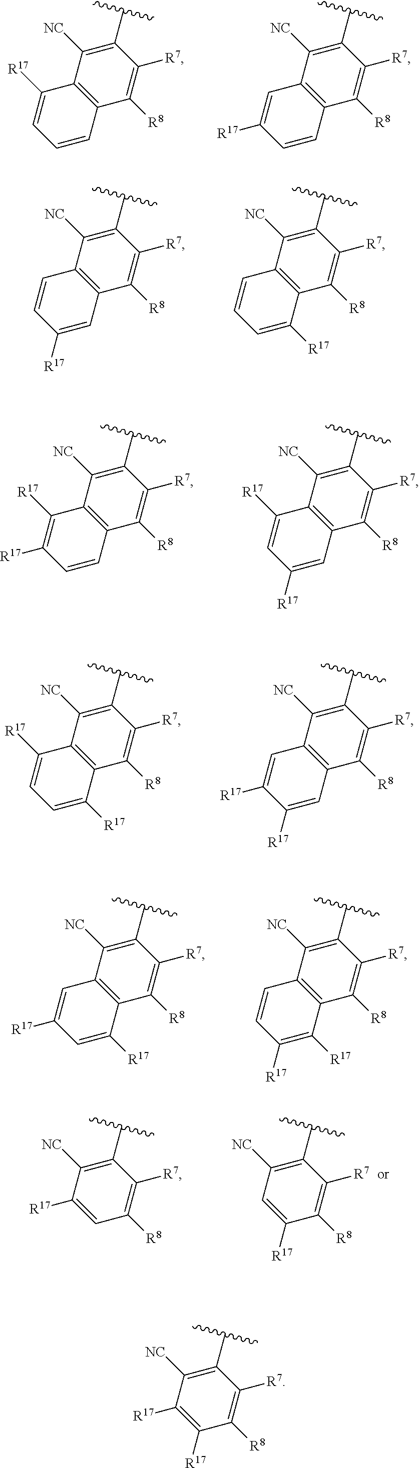

Aspect 31. The compound of any one of the preceding Aspects, wherein the

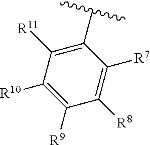

moiety is

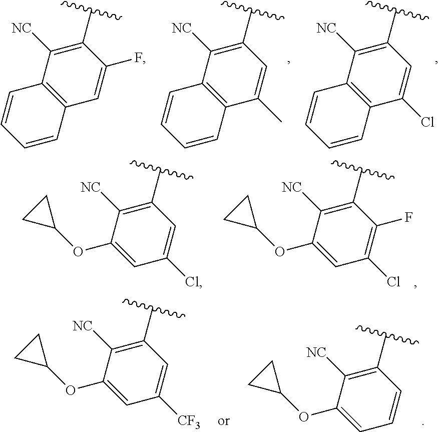

Aspect 32. The compound of any one of the preceding Aspects, wherein the compound is selected from

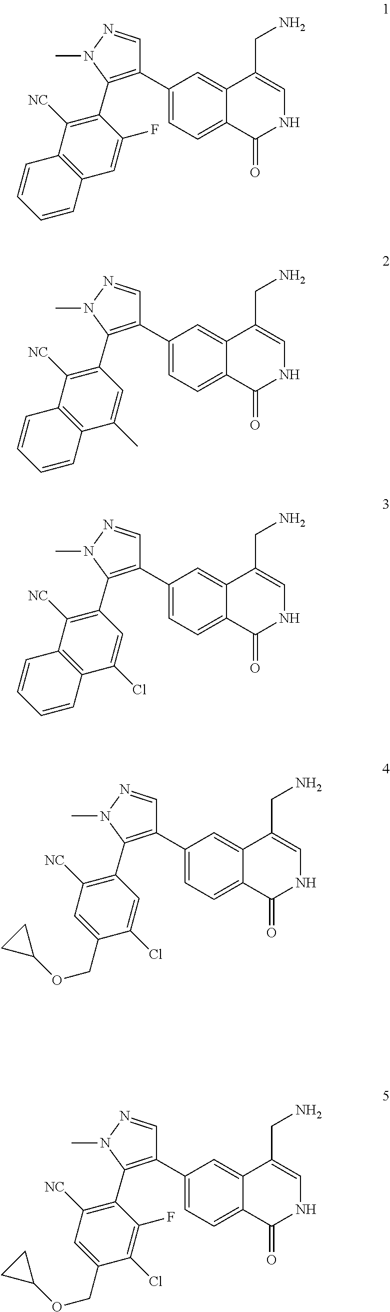 [SMILES: Cn1ncc(-c2ccc3c(=O)[nH]cc(CN)c3c2)c1-c1c(F)cc2ccccc2c1C#N ; Cc1cc(-c2c(-c3ccc4c(=O)[nH]cc(CN)c4c3)cnn2C)c(C#N)c2ccccc12 ; Cn1ncc(-c2ccc3c(=O)[nH]cc(CN)c3c2)c1-c1cc(Cl)c2ccccc2c1C#N ; Cn1ncc(-c2ccc3c(=O)[nH]cc(CN)c3c2)c1-c1cc(Cl)c(COC2CC2)cc1C#N ; Cn1ncc(-c2ccc3c(=O)[nH]cc(CN)c3c2)c1-c1c(C#N)cc(COC2CC2)c(Cl)c1F]

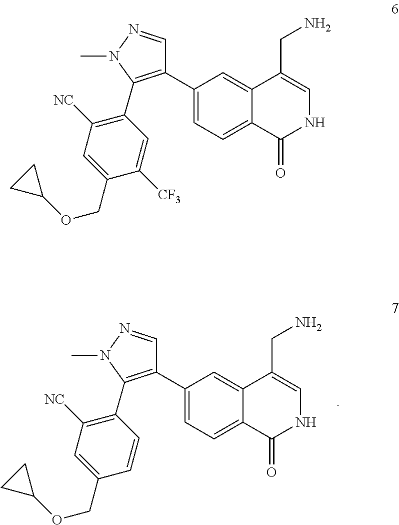 [SMILES: Cn1ncc(-c2ccc3c(=O)[nH]cc(CN)c3c2)c1-c1cc(C(F)(F)F)c(COC2CC2)cc1C#N ; Cn1ncc(-c2ccc3c(=O)[nH]cc(CN)c3c2)c1-c1ccc(COC2CC2)cc1C#N]

Aspect 33. A pharmaceutical composition comprising a compound of any one of Aspects 1-32 or a pharmaceutically acceptable salt, stereoisomer, tautomer or prodrug thereof, together with a pharmaceutically acceptable excipient.

Aspect 34. A method of decreasing PRMT5 activity by inhibition, which comprises administering to an individual the compound according to any one of Aspects 1-33, or a pharmaceutically acceptable salt thereof, including the compound of formula (I) or the specific compounds exemplified herein.

Aspect 35. The method of Aspect 34, wherein the disease is selected from cancer.

Aspect 36. Use of a compound of any one of Aspects 1-32 or a pharmaceutically acceptable salt, stereoisomer, tautomer or prodrug thereof in the preparation of a medicament for treating a disease that is modulated by PRMT5.

Aspect 37. The use of Aspect 36, wherein the disease is cancer.

Aspect 38. The use of Aspect 37, wherein the disease is MTAP-null solid tumor, including but not limited to lung cancer, bladder cancer, melanoma, pancreatic cancer, esophageal cancer, gastric adenocarcinoma, breast cancer, glioblastoma, etc.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2] nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur(S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113 (3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)-groups (keto forms) may undergo tautomerism to form hydroxyl —CH═C(OH)-groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### EXAMPLES

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art. In the synthesis schemes, some protection/deprotection steps are not shown and can be incorporated before, after or in between any steps.

Reactions can be monitored according to any suitable method known in the art, such as NMR, UV, HPLC, LC-MS and TLC. Compounds can be purified by a variety of methods, including prep-HPLC and silica gel chromatography. Unless specified, prep-HPLC uses a buffered acetonitrile/water systems and silica gel chromatography (including column chromatography and prep-TLC) uses PE/EtOAc or DCM/MeOH systems as mobile phases.

Chiral analytic HPLC was used for the retention time analysis of different chiral examples, the conditions were divided into the methods as below according to the column, mobile phase, solvent ratio used.

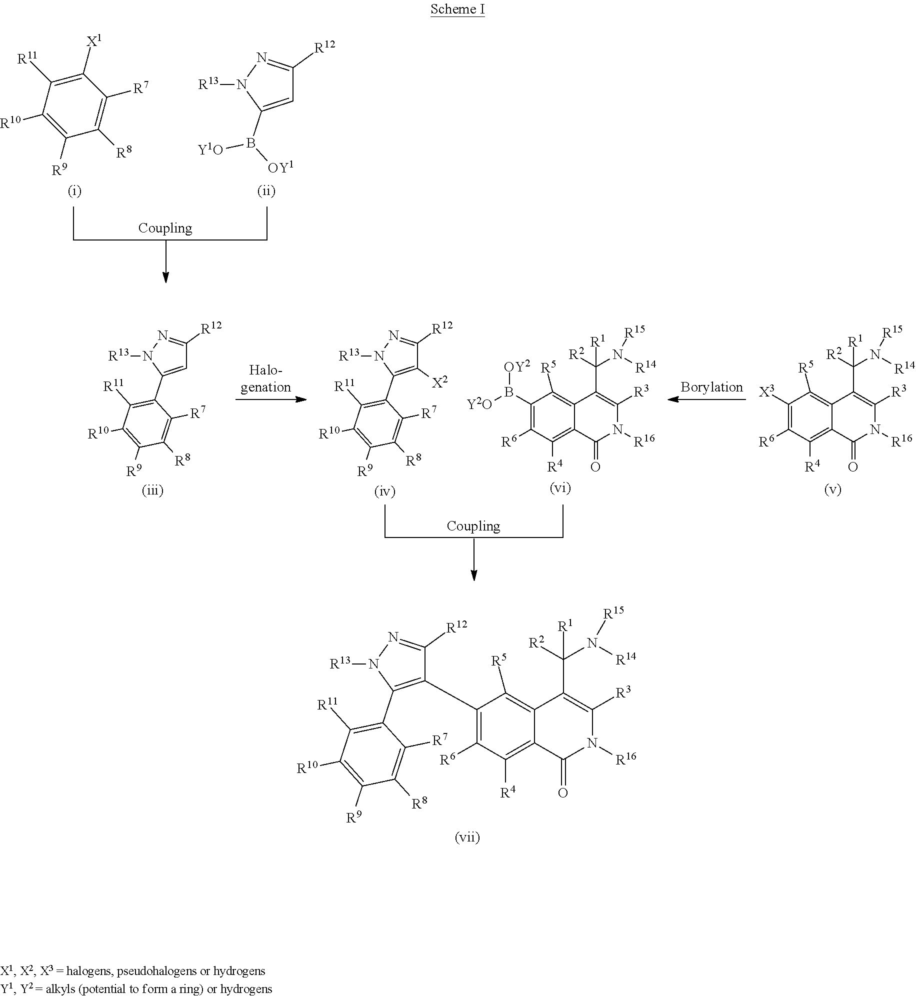

For example, compounds of Formula (I) can be formed as shown in Scheme I. Compound (i) and compound (ii) can be coupled via transition metal catalyzed reactions to give compound (iii); compound (iii) can be halogenated to give compound (iv). In parallel, compound (vi) can be borylated to give compound (vii). Compound (iv) and compound (vi) can be coupled via transition metal catalyzed reactions to give compound (vii) [i.e., Formula (I)].

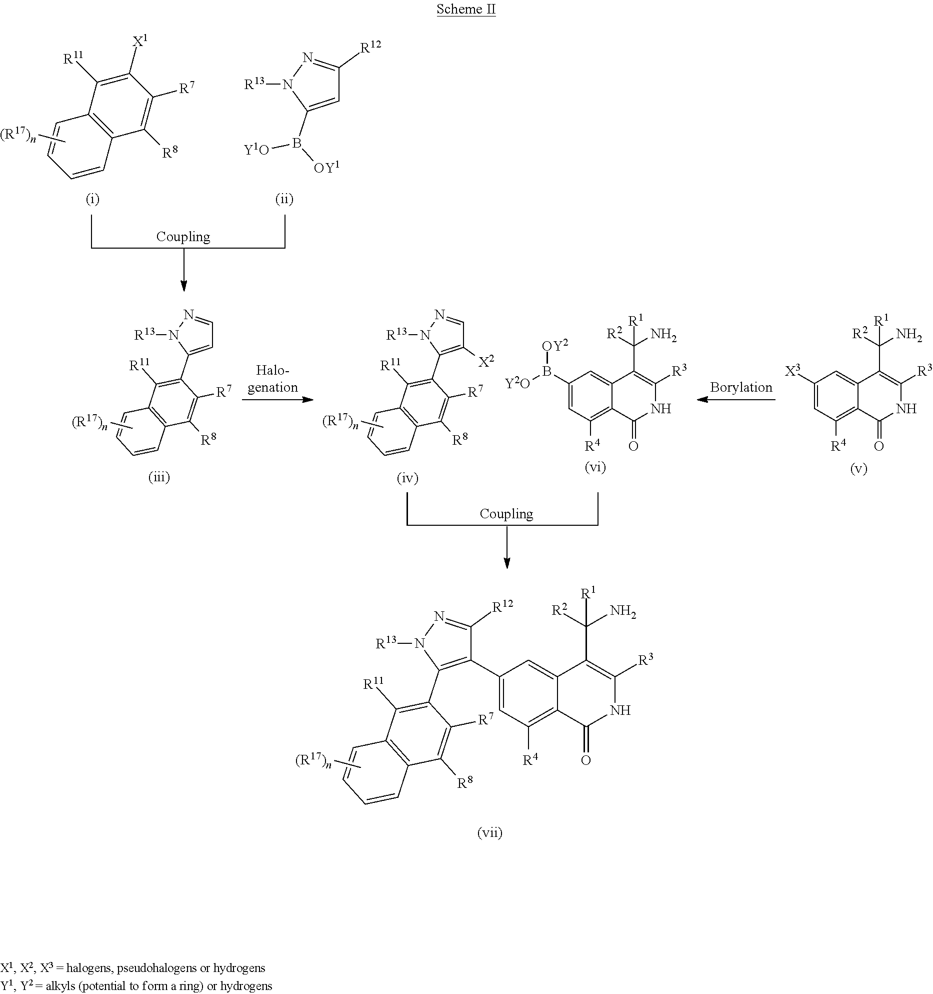

For example, compounds of Formula (I) can be formed as shown in Scheme I. Compound (i) and compound (ii) can be coupled via transition metal catalyzed reactions to give compound (iii); compound (iii) can be halogenated to give compound (iv). In parallel, compound (vi) can be borylated to give compound (vii). Compound (iv) and compound (vi) can be coupled via transition metal catalyzed reactions to give compound (vii) [i.e., Formula (IIIa)].

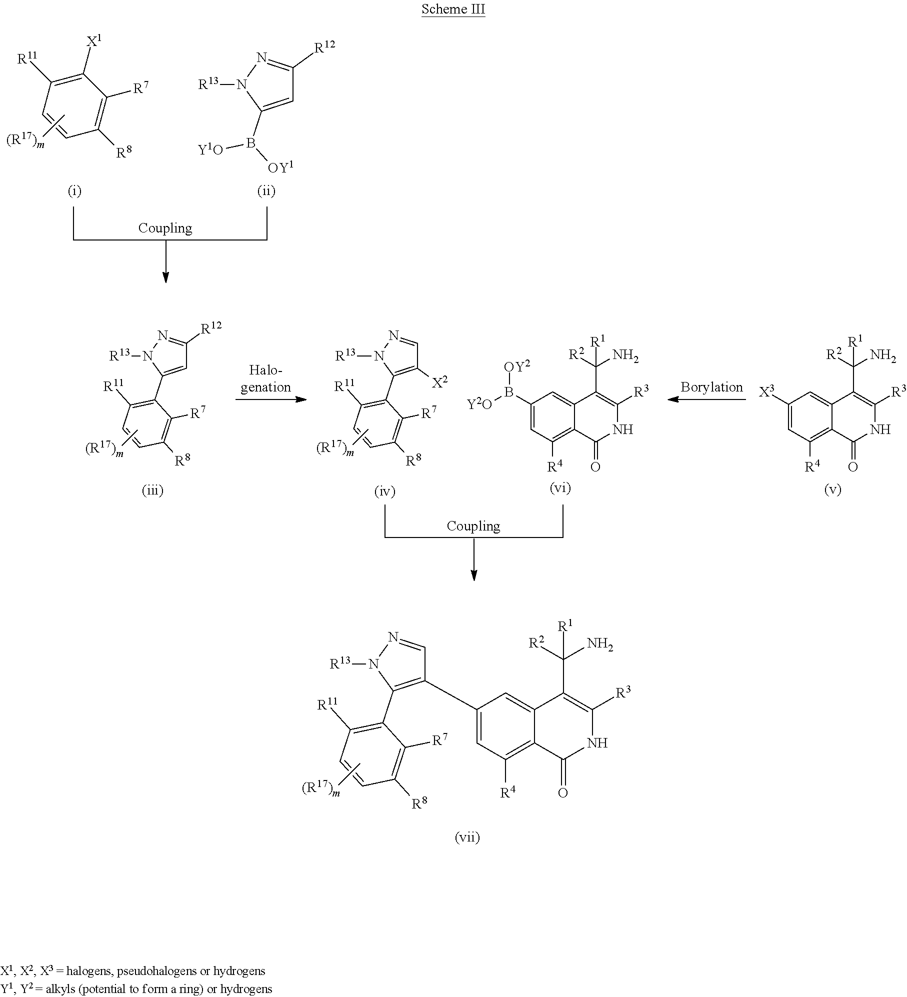

For example, compounds of Formula (I) can be formed as shown in Scheme I. Compound (i) and compound (ii) can be coupled via transition metal catalyzed reactions to give compound (iii); compound (iii) can be halogenated to give compound (iv). In parallel, compound (vi) can be borylated to give compound (vii). Compound (iv) and compound (vi) can be coupled via transition metal catalyzed reactions to give compound (vii) [i.e., Formula (IIIb)].

### Abbreviations

NMR nuclear magnetic resonance
UV ultraviolet
HPLC high performance liquid chromatography
LC-MS liquid chromatograph mass spectrometer
TLC thin layer chromatography
PE petroleum ether
Et ethyl
Ac acetyl
DCM dichloromethane
Me methyl
DMSO dimethyl sulfoxide
Boc tert-butyloxycarbonyl
dppf 1,1′-bis(diphenylphosphino) ferrocene
BPD bis(pinacolato)diboron
Bu butyl
m-CPBA meta-chloroperbenzoic acid
dba dibenzylideneacetone
DMF N,N-dimethylformamide
THF tetrahydrofuran
dtbpf 1,1′-bis(di-tert-butylphosphino) ferrocene
NBS N-bromosuccinimide
NCS N-iodosuccinimide
DCE dichloroethane
Ts p-toluenesulfonyl
MTBE methyl tert-butyl ether
TR-FRET time-resolved fluorescence resonance energy transfer
tris-HCl tris(hydroxymethyl)aminomethane hydrochloride
BSA bovine serum albumin
TCEP tris(2-carboxyethyl) phosphine

### Example 1: 2-(4-(4-(aminomethyl)-1-oxo-1,2-dihydroisoquinolin-6-yl)-1-methyl-1H-pyrazol-5-yl)-3-fluoro-1-naphthonitrile

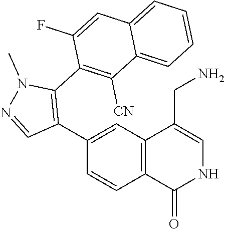 [SMILES: Cn1ncc(-c2ccc3c(=O)[nH]cc(CN)c3c2)c1-c1c(F)cc2ccccc2c1C#N]

### Step 1: methyl 4-bromo-2-(dicyanomethyl)benzoate

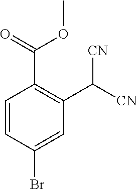 [SMILES: COC(=O)c1ccc(Br)cc1C(C#N)C#N]

A mixture of methyl 4-bromo-2-iodobenzoate (6.0 g, 17.6 mmol), malononitrile (1.4 g, 21.2 mmol), (S)-proline (400 mg, 3.5 mmol), CuI (660 mg, 3.5 mmol) and K₂CO₃ (3.6 g, 26.1 mmol) in DMSO (60 mL) was heated to 60° C. and stirred for 3 h. The mixture was cooled to room temperature, diluted with water (300 mL) then concentrated HCl was added to adjust the pH of the mixture to 5˜6. The mixture was extracted with EtOAc (200 mL×3). The combined organic layer was washed with brine (200 mL), dried over Na₂SO₄, filtered and concentrated in vacuo. The crude was purified with silica gel column chromatography (PE:EtOAc=4:1) to give the title compound (3.7 g, 76%). LC-MS (M+H)⁺=278.9.

### Step 2: tert-butyl ((6-bromo-1-oxo-1,2-dihydroisoquinolin-4-yl)methyl)carbamate

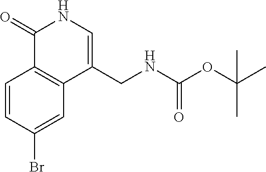 [SMILES: CC(C)(C)OC(=O)NCc1c[nH]c(=O)c2ccc(Br)cc12]

To a solution of methyl 4-bromo-2-(dicyanomethyl)benzoate (500 mg, 1.8 mmol) in ethanol (10 mL) was added Raney Ni (100 mg) and ammonia solution (25%, 2 mL). The mixture was heated to 50° C. under 4 atm of hydrogen overnight. Solids were filtered off and solvent was evaporated in vacuo. The residue was dissolved in DCM (10 mL), then triethylamine (0.5 mL, 12.2 mmol) and Boc₂O (780 mg, 9.1 mmol) was added. The mixture was stirred at room temperature overnight then concentrated under reduced pressure. The crude was purified by silica gel column chromatography (PE:EtOAc=10:1) to give the title compound (50 mg, 8%). LC-MS (M+H)⁺=353.0.

### Step 3: tert-butyl ((1-oxo-6-(4,4,5,5-tetramethyl-1,3,2-dioxaborolan-2-yl)-1,2-dihydroisoquinolin-4-yl)methyl)carbamate

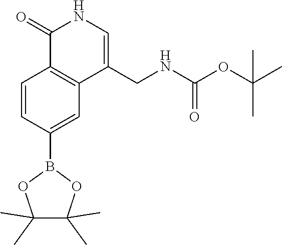 [SMILES: CC(C)(C)OC(=O)NCc1c[nH]c(=O)c2ccc(B3OC(C)(C)C(C)(C)O3)cc12]

To a solution of tert-butyl ((6-bromo-1-oxo-1,2-dihydroisoquinolin-4-yl)methyl)carbamate (100 mg, 0.28 mmol) in dioxane (1 mL) was added Pd(dppf)Cl₂ (20.72 mg, 0.028 mmol), BPD (79 mg, 0.31 mmol) and KOAc (83 mg, 0.85 mmol). The mixture was heated to 100° C. and stirred for 12 h. The mixture was cooled to room temperature and concentrated under reduced pressure. The residue was purified by prep-TLC to give the title compound (53 mg, 46%). LC-MS (M+H)⁺=401.0.

### Step 4: 5-bromo-7-fluoro-9-methyl-1,4-dihydro-1,4-epiminonaphthalene

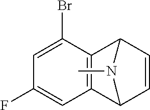

To a solution of 1,3-dibromo-2-chloro-5-fluorobenzene (30.5 g, 105.9 mmol) and 1-methylpyrrole (17.2 g, 211.8 mmol) in toluene (750 mL) was added n-BuLi (2.5 M in hexane, 44.5 mL, 111 mol) dropwise at −30° C. under nitrogen. The mixture was stirred for 30 min at −30° C. and then allowed to warm to room temperature. After 12 h, the mixture was quenched with water (10 mL) and concentrated under reduced pressure. The residue was diluted with EtOAc (500 mL), washed with brine (500 mL), dried over Na₂SO₄, filtered and concentrated under vacuum. The residue was purified by silica gel chromatography (PE:EtOAc=3:2) to give the title compound (15 g, 60%). LC-MS (M+H)⁺=254.1.

### Step 5: 1-bromo-3-fluoronaphthalene

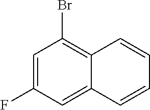 [SMILES: Fc1cc(Br)c2ccccc2c1]

To a solution of 5-bromo-7-fluoro-9-methyl-1,4-dihydro-1,4-epiminonaphthalene (9.3 g, 36.8 mmol) in chloroform (250 mL) was carefully added m-CPBA (75%, 17 g, 73.5 mmol) in portions whilst maintaining the inner temperature below 40° C. After 26 h, solid was filtered off and the filtrate was concentrated under reduced pressure. The residue was purified by silica gel chromatography to give the title compound (7.3 g, 89%). LC-MS (M+H)⁺=225.1.

### Step 6: 3-fluoro-1-naphthonitrile

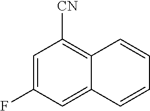 [SMILES: N#Cc1cc(F)cc2ccccc12]

A mixture of 1-bromo-3-fluoronaphthalene (4.4 g, 19.6 mmol), Pd₂ (dba) 3 (1.8 g, 1.96 mmol), zinc cyanide (5.7 g, 49 mmol), dppf (2.2 g, 3.9 mmol) and Zn power (127 mg, 1.96 mmol) in DMF (50 mL) was purged with N₂ and then stirred for 4 h at 115° C. The mixture was cooled to room temperature, filtered, and the filtrate was diluted with EtOAc (200 mL), washed with brine (200 mL), dried over Na₂SO₄, filtered and concentrated under vacuum. The residue was purified by silica gel chromatography (PE:EtOAc=10:1) to give the title compound (3.1 g, 92%). LC-MS (M+H)⁺=172.1.

### Step 7: 3-fluoro-2-iodo-1-naphthonitrile

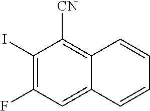 [SMILES: N#Cc1c(I)c(F)cc2ccccc12]

n-BuLi (2.5 M in hexane, 7.7 mL, 19.2 mol) was added to a solution of N-isopropylpropan-2-amine (2.3 g, 22.7 mmol) in THF (50 mL) at −78° C. and stirred for 15 min. 3-fluoro-1-naphthonitrile (3.0 g, 17.4 mmol) in THF (40 mL) was added to the mixture and stirred for 30 min at −78° C. Then a solution of iodine (5.7 g, 22.7 mmol) in THF (40 mL) was added to the mixture and stirred for 30 min at −78° C. The mixture was warmed to room temperature and stirred for another 10 h. The mixture was quenched with saturated NH₄Cl (40 mL) and extracted with EtOAc (150 mL). The organic layer was washed with brine (150 mL), dried over Na₂SO₄, filtered and concentrated under vacuum. The residue was purified by silica gel column (PE:EtOAc=10:1) to give the title compound (2.6 g, 50%).

### Step 8: 3-fluoro-2-(1-methyl-1H-pyrazol-S-yl)-1-naphthonitrile

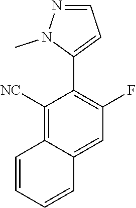 [SMILES: Cn1nccc1-c1c(F)cc2ccccc2c1C#N]

To a solution of 3-fluoro-2-iodo-1-naphthonitrile (1.6 g, 5.4 mmol) and 1-methyl-5-(4,4,5,5-tetramethyl-1,3,2-dioxaborolan-2-yl)-1H-pyrazole (2.5 g, 11.9 mmol) in dioxane (20 mL) and water (2 mL) was added Pd(dtbpf)Cl₂ (352 mg, 0.54 mmol) and NaHCO₃ (1.4 g, 16.2 mmol), then stirred for 12 h at 80° C. The mixture was cooled to room temperature, diluted with EtOAc (100 mL), successively washed with water (50 mL), brine (50 mL), dried over Na₂SO₄, filtered and concentrated under vacuum. The residue was purified by silica gel chromatography (PE:EtOAc=2:1) to give the title compound (840 mg, 62%). LC-MS (M+H)⁺=252.2.

### Step 9: 2-(4-bromo-1-methyl-1H-pyrazol-5-yl)-3-fluoro-1-naphthonitrile

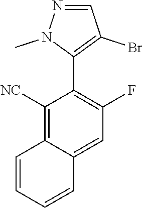 [SMILES: Cn1ncc(Br)c1-c1c(F)cc2ccccc2c1C#N]

To a solution of 3-fluoro-2-(1-methyl-1H-pyrazol-5-yl)-1-naphthonitrile (560 mg, 2.2 mmol) in MeCN (10 mL) was added NBS (713 mg, 4.0 mmol) and the mixture was stirred for 3 h at room temperature. The mixture was diluted with EtOAc (30 mL), successively washed with water (20 mL), brine (20 mL), dried over Na₂SO₄, filtered and concentrated under vacuum. The residue was purified by silica gel chromatography (PE:EtOAc=3:1) to give the title compound (640 mg, 87%). LC-MS (M+H)⁺=330.1.

### Step 10: tert-butyl ((6-(5-(1-cyano-3-fluoronaphthalen-2-yl)-1-methyl-1H-pyrazol-4-yl)-1-oxo-1,2-dihydroisoquinolin-4-yl)methyl)carbamate

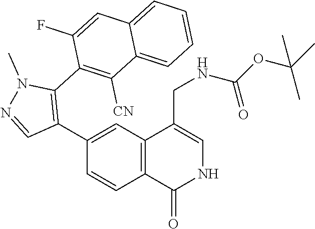 [SMILES: Cn1ncc(-c2ccc3c(=O)[nH]cc(CNC(=O)OC(C)(C)C)c3c2)c1-c1c(F)cc2ccccc2c1C#N]

A mixture of tert-butyl ((1-oxo-6-(4,4,5,5-tetramethyl-1,3,2-dioxaborolan-2-yl)-1,2-dihydroisoquinolin-4-yl)methyl)carbamate, 2-(4-bromo-1-methyl-1H-pyrazol-5-yl)-3-fluoro-1-naphthonitrile (53 mg, 0.14 mmol), Pd(dppf)Cl₂ (10 mg, 0.01 mmol), K₂CO₃ (39 mg, 0.28 mmol) in dioxane (5 mL) and water (1 mL) was heated to reflux for 8 h under nitrogen. The mixture was cooled to room temperature, then partitioned between water (20 mL) and EtOAc (20 mL). The separated organic layer was dried over Na₂SO₄, filtered and concentrated in vacuo to give the title compound (50 mg, 33%). LC-MS (M+H)⁺=524.2.

### Step 11: 2-(4-(4-(aminomethyl)-1-oxo-1,2-dihydroisoquinolin-6-yl)-1-methyl-1H-pyrazol-5-yl)-3-fluoro-1-naphthonitrile

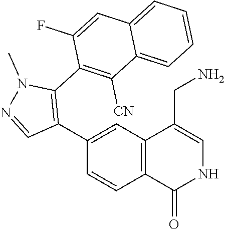 [SMILES: Cn1ncc(-c2ccc3c(=O)[nH]cc(CN)c3c2)c1-c1c(F)cc2ccccc2c1C#N]

To solid tert-butyl ((6-(5-(1-cyano-3-fluoronaphthalen-2-yl)-1-methyl-1H-pyrazol-4-yl)-1-oxo-1,2-dihydroisoquinolin-4-yl)methyl)carbamate (50 mg, 0.96 mmol) was added in HCl in dioxane (4.0 M, 6 mL). The mixture was stirred at room temperature for 2 h then concentrated in vacuo. The crude was purified by prep-TLC to give Example 1 (6 mg, 15%). LC-MS (M+H)⁺=424.2. ¹H NMR (400 MHZ, DMSO-d6) δ 11.06 (s, 1H), 8.51 (d, J=10.2 Hz, 1H), 8.24 (d, J=7.0 Hz, 2H), 8.14 (d, J=8.5 Hz, 1H), 8.05 (d, J=8.3 Hz, 1H), 7.89-7.84 (m, 2H), 7.41 (s, 1H), 7.35 (d, J=8.4 Hz, 1H), 6.97 (s, 1H), 3.80 (s, 3H), 3.31 (s, 2H).

### Example 2: 2-(4-(4-(aminomethyl)-1-oxo-1,2-dihydroisoquinolin-6-yl)-1-methyl-1H-pyrazol-5-yl)-4-chloro-6-cyclopropoxybenzonitrile hydrochloride

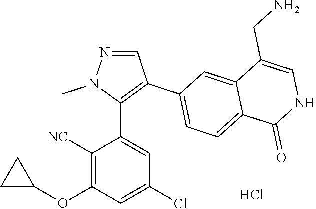 [SMILES: Cn1ncc(-c2ccc3c(=O)[nH]cc(CN)c3c2)c1-c1cc(Cl)cc(OC2CC2)c1C#N ; Cl]

### Step 1: 2-bromo-4-chloro-6-fluorobenzonitrile

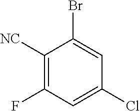 [SMILES: N#Cc1c(F)cc(Cl)cc1Br]

To a solution of 2-bromo-4-chloro-6-fluoroaniline (15 g, 67 mmol) in anhydrous DCM (60 mL) was added nitrosonium tetrafluoroborate (8.6 g, 74 mmol) and the mixture was stirred at 25° C. for 1 h. The reaction mixture was then cooled to 0° C. KCN (9.3 g, 143 mmol) was added to the mixture followed by dropwise addition of CuSO₄·5H₂O (33 g, 134 mmol) in water (300 mL). After stirring for 40 minutes at 0° C., the reaction mixture was allowed to warm to 25° C. and stirred for another 1 h. The reaction mixture was diluted with DCM (100 mL) and quenched with slow addition of saturated NaHCO₃ solution until gas evolution ceased. The heterogeneous mixture was filtered through a pad of celite. the organic layer was separated, washed with brine (50 mL×2), dried over Na₂SO₄, and concentrated. The residue was purified by silica gel column chromatography (PE:EtOAc=1:0 to 50:1) to give the title compound (3.7 g, 24%).

### Step 2: 2-bromo-4-chloro-6-cyclopropoxybenzonitrile

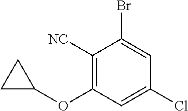 [SMILES: N#Cc1c(Br)cc(Cl)cc1OC1CC1]

To a solution of 2-bromo-4-chloro-6-fluorobenzonitrile (3.7 g, 16 mmol), cyclopropanol (1.37 g, 23.7 mmol) in DMF (15 mL) was added Cs₂CO₃ (12.9 g, 39.5 mmol). The mixture was stirred at 25° C. for 4 h, then quenched with water (45 mL). The mixture was extracted with EtOAc (45 mL×3). The combined organic layer was washed with brine (50 mL×2), dried over Na₂SO₄, filtered and concentrated under vacuum. The residue was purified by silica gel column chromatography (PE:EtOAc=1:0 to 1:1) to give the title compound (1.5 g, 35%). LC-MS (M+H)⁺=272.0.

### Step 3: 4-chloro-2-cyclopropoxy-6-(1-methyl-1H-pyrazol-5-yl)benzonitrile

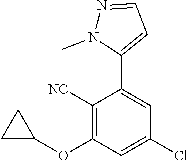 [SMILES: Cn1nccc1-c1cc(Cl)cc(OC2CC2)c1C#N]

To a solution of 2-bromo-4-chloro-6-cyclopropoxybenzonitrile (1.3 g, 4.77 mmol), 1-methyl-5-(4,4,5,5-tetramethyl-1,3,2-dioxaborolan-2-yl) pyrazole (993 mg, 4.77 mmol) in dioxane (1 mL) and water (0.2 mL) was added NaHCO₃ (1.0 g, 12 mmol) and Pd (t-Bu₃P)₂ (244 mg, 0.48 mmol). The mixture was stirred at 80° C. for 12 h. The mixture was cooled to room temperature and concentrated under vacuum. The residue was purified by silica gel column chromatography (PE:EtOAc=1:0 to 0:1) to give the title compound (0.4 g, 31%). ¹H NMR (400 MHz, CDCl₃) δ 7.55 (d, J=1.6 Hz, 1H), 7.41 (d, J=1.2 Hz, 1H), 7.03 (d, J=1.6 Hz, 1H), 6.45 (d, J=1.6 Hz, 1H), 3.96-3.81 (m, 4H), 3.74 (s, 3H), 1.01-0.88 (m, 4H), 0.88-0.75 (m, 2H). LC-MS (M+H)⁺=274.1.

### Step 4: 4-chloro-2-cyclopropoxy-6-(4-iodo-1-methyl-1H-pyrazol-5-yl)benzonitrile

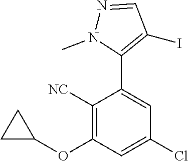 [SMILES: Cn1ncc(I)c1-c1cc(Cl)cc(OC2CC2)c1C#N]

To a solution 4-chloro-2-cyclopropoxy-6-(1-methyl-1H-pyrazol-5-yl)benzonitrile (0.35 g, 1.28 mmol) in AcOH (3.5 mL) was added NIS (575 mg, 2.56 mmol). The mixture was stirred at 60° C. for 4 h. To the mixture was added PE (5.0 mL), then the precipitate was collected by filtration. The solid was washed by PE (5 mL) to give the title compound (0.10 g, 20%). LC-MS (M+H)⁺=400.0.

### Step 5: tert-butyl ((6-(5-(5-chloro-2-cyano-3-cyclopropoxyphenyl)-1-methyl-1H-pyrazol-4-yl)-1-oxo-1,2-dihydroisoquinolin-4-yl)methyl)carbamate

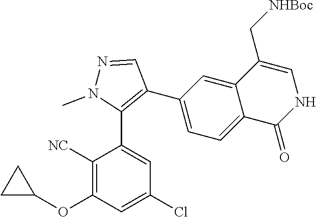 [SMILES: Cn1ncc(-c2ccc3c(=O)[nH]cc(CNC(=O)OC(C)(C)C)c3c2)c1-c1cc(Cl)cc(OC2CC2)c1C#N]

To a solution of 4-chloro-2-cyclopropoxy-6-(4-iodo-1-methyl-1H-pyrazol-5-yl)benzonitrile (80 mg, 0.20 mmol) and tert-butyl ((1-oxo-6-(4,4,5,5-tetramethyl-1,3,2-dioxaborolan-2-yl)-1,2-dihydroisoquinolin-4-yl)methyl)carbamate (40 mg, 0.10 mmol) in dioxane (1 mL) and water (0.2 mL) was added Na₂CO₃ (42 mg, 0.40 mmol) and Pd(dppf)Cl₂ (15 mg, 0.02 mmol). The mixture was stirred at 80° C. for 4 h. The reaction mixture was cooled to room temperature and concentrated under reduced pressure. The residue was purified by prep-TLC to give the title compound (20 mg, 37%). LC-MS (M+H)⁺=546.2.

### Step 6: 2-(4-(4-(aminomethyl)-1-oxo-1,2-dihydroisoquinolin-6-yl)-1-methyl-1H-pyrazol-5-yl)-4-chloro-6-cyclopropoxybenzonitrile hydrochloride

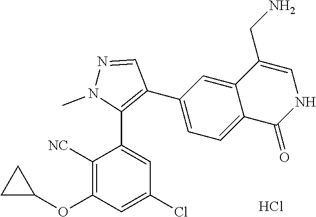 [SMILES: Cn1ncc(-c2ccc3c(=O)[nH]cc(CN)c3c2)c1-c1cc(Cl)cc(OC2CC2)c1C#N ; Cl]

To a solution of tert-butyl ((6-(5-(5-chloro-2-cyano-3-cyclopropoxyphenyl)-1-methyl-1H-pyrazol-4-yl)-1-oxo-1,2-dihydroisoquinolin-4-yl)methyl)carbamate (20 mg, 0.037 mmol) in MeOH (0.5 mL) was added HCl/MeOH (4 M, 0.3 mL). The mixture was stirred at 25° C. for 0.5 h, then concentrated under reduced pressure. The crude was purified by prep-HPLC with water (HCl)/MeCN system to give Example 2 (7 mg, 38%). ¹H NMR (400 MHz, DMSO-d6) δ 11.50-11.40 (m, 1H), 8.48-8.20 (m, 4H), 8.06 (d, J=8.4 Hz, 1H), 7.82-7.73 (m, 2H), 7.49-7.43 (m, 1H), 7.40 (d, J=6.0 Hz, 1H), 7.02 (d, J=8.4 Hz, 1H), 4.27-4.19 (m, 1H), 4.17-4.00 (m, 2H), 3.74 (s, 3H), 0.99-0.88 (m, 2H), 0.88-0.75 (m, 2H). LC-MS (M−NH₂)⁺=429.0.

### Example 3: 2-(4-(4-(aminomethyl)-1-oxo-1,2-dihydroisoquinolin-6-yl)-1-methyl-1H-pyrazol-5-yl)-4-chloro-6-cyclopropoxy-3-fluorobenzonitrile hydrochloride

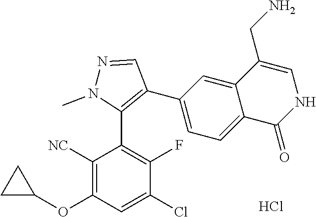 [SMILES: Cn1ncc(-c2ccc3c(=O)[nH]cc(CN)c3c2)c1-c1c(F)c(Cl)cc(OC2CC2)c1C#N ; Cl]

### Step 1: 4-chloro-2-cyclopropoxy-5-fluorobenzonitrile

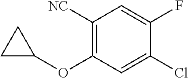 [SMILES: N#Cc1cc(F)c(Cl)cc1OC1CC1]

To a solution of 4-chloro-2,5-difluoro-benzonitrile (45.0 g, 259 mmol), cyclopropanol (18.0 g, 311 mmol) in DMF (150 mL) was added Cs₂CO₃ (168 g, 518 mmol). The mixture was stirred at 25° C. for 12 h. The mixture was quenched by water (1500 mL) and extracted with EtOAc (500 mL×3). The combined organic layer was washed with brine (100 mL×2), dried over Na₂SO₄, filtered and concentrated under vacuum. The residue was purified by silica gel column chromatography (PE:EtOAc=1:0 to 1:1) to give the title compound (70.0 g, 64%).

### Step 2: 4-chloro-6-cyclopropoxy-3-fluoro-2-iodobenzonitrile

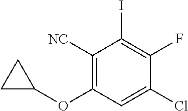 [SMILES: N#Cc1c(OC2CC2)cc(Cl)c(F)c1I]

To a solution of 4-chloro-2-cyclopropoxy-5-fluorobenzonitrile (68.5 g, 323 mmol) in THF (316 mL) was added LDA (2.0 M in THF/heptane, 194 mL) dropwise at −78° C. over 0.5 h. Iodine (98.5 g, 388 mmol) in THF (95.0 mL) was added dropwise at −78° C. over 1.5 h. The residue was poured into saturated NH₄Cl (100 mL) and stirred for 30 mins. The mixture was extracted with ethyl acetate (150 mL×3). The combined organic layer was washed with 10% Na₂SO₃ (150 mL), brine (50.0 mL×2), dried over Na₂SO₄, filtered and concentrated under vacuum. The residue was purified by silica gel column chromatography (PE:EtOAc=1:0 to 1:1) to give the title compound (60.0 g, 55%).

### Step 3: 4-chloro-6-cyclopropoxy-3-fluoro-2-(1-methyl-1H-pyrazol-5-yl)benzonitrile

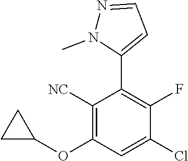 [SMILES: Cn1nccc1-c1c(F)c(Cl)cc(OC2CC2)c1C#N]

To a mixture of 4-chloro-6-cyclopropoxy-3-fluoro-2-iodobenzonitrile (50.0 g, 148 mmol), 1-methyl-5-(4,4,5,5-tetramethyl-1,3,2-dioxaborolan-2-yl)-1H-pyrazole (40.0 g, 192 mmol) and KF (21.5 g, 370 mmol) in dioxane (250 mL) and water (50 mL) was added bis-(di-tert-butyl (4-dimethylaminophenyl)phosphine)dichloropalladium (10.4 g, 14.8 mmol). The mixture was stirred at 60° C. for 16 h. The mixture was poured into water (150 mL) and extracted with EtOAc (500 mL×2). The combined organic layer was concentrated in vacuum. The residue was purified by silica gel column chromatography (PE:EtOAc=20:1 to 3:1) to give the title compound (30.0 g, 49%). LC-MS (M+H)⁺=292.0.

### Step 4: 4-chloro-6-cyclopropoxy-3-fluoro-2-(4-iodo-1-methyl-1H-pyrazol-5-yl)benzonitrile

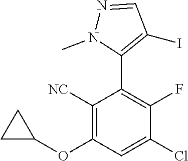 [SMILES: Cn1ncc(I)c1-c1c(F)c(Cl)cc(OC2CC2)c1C#N]

To a solution of 4-chloro-6-cyclopropoxy-3-fluoro-2-(1-methyl-1H-pyrazol-5-yl)benzonitrile (30.0 g, 102 mmol) in AcOH (180 mL) was added NIS (46.2 g, 205 mmol). The mixture was stirred at 60° C. for 3.5 h. The mixture was cooled to room temperature and concentrated under reduced pressure. The residue was purified by silica gel column chromatography (PE:EtOAc=20:1 to 3:1) to give the title compound (24.5 g, 57%). LC-MS (M+H)⁺=418.0.

### Step 5: tert-butyl ((6-(5-(3-chloro-6-cyano-5-cyclopropoxy-2-fluorophenyl)-1-methyl-1H-pyrazol-4-yl)-1-oxo-1,2-dihydroisoquinolin-4-yl)methyl)carbamate

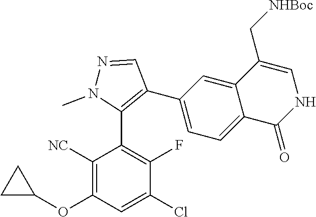 [SMILES: Cn1ncc(-c2ccc3c(=O)[nH]cc(CNC(=O)OC(C)(C)C)c3c2)c1-c1c(F)c(Cl)cc(OC2CC2)c1C#N]

To a solution of 4-chloro-6-cyclopropoxy-3-fluoro-2-(4-iodo-1-methyl-1H-pyrazol-5-yl)benzonitrile (20 mg, 0.048 mmol), tert-butyl ((1-oxo-6-(4,4,5,5-tetramethyl-1,3,2-dioxaborolan-2-yl)-1,2-dihydroisoquinolin-4-yl)methyl)carbamate (20 mg, 0.048 mmol) in dioxane (1 mL) and water (0.2 mL) was added Na₂CO₃ (10 mg, 0.096 mmol) and Pd(dppf)Cl₂ (3.5 mg, 0.005 mmol). The mixture was stirred at 80° C. for 4 h. The reaction mixture was cooled to room temperature and concentrated under reduced pressure. The residue was purified by prep-TLC to give the title compound (20 mg, 74%). LC-MS (M+H)⁺=564.3.

### Step 6: 2-(4-(4-(aminomethyl)-1-oxo-1,2-dihydroisoquinolin-6-yl)-1-methyl-1H-pyrazol-5-yl)-4-chloro-6-cyclopropoxy-3-fluorobenzonitrile hydrochloride

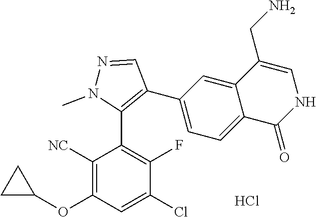 [SMILES: Cn1ncc(-c2ccc3c(=O)[nH]cc(CN)c3c2)c1-c1c(F)c(Cl)cc(OC2CC2)c1C#N ; Cl]

To a solution of tert-butyl ((6-(5-(3-chloro-6-cyano-5-cyclopropoxy-2-fluorophenyl)-1-methyl-1H-pyrazol-4-yl)-1-oxo-1,2-dihydroisoquinolin-4-yl)methyl)carbamate (20 mg, 0.035 mmol) in MeOH (0.5 mL) was added HCl in MeOH (4.0 M, 4.0 mL). The mixture was stirred at 25° C. for 0.5 h then concentrated under reduced pressure. The crude was purified by prep-HPLC with water (HCl)/MeCN system to give Example 3 (7 mg, 45%). ¹H NMR (400 MHZ, DMSO-d6) δ 11.48 (d, J=5.6 Hz, 1H), 8.41 (s, 1H), 8.40-8.20 (br s, 3H), 8.08 (d, J=8.4 Hz, 1H), 8.01 (d, J=6.0 Hz, 1H), 7.83 (s, 1H), 7.41 (d, J=6.0 Hz, 1H), 7.04 (d, J=8.4 Hz, 1H), 4.28-4.22 (m, 1H), 4.20-4.02 (m, 2H), 3.77 (s, 3H), 0.98-0.87 (m, 2H), 0.87-0.76 (m, 2H). LC-MS (M−NH₂)⁺=447.0.

### Example 4: 2-(4-(4-(aminomethyl)-1-oxo-1,2-dihydroisoquinolin-6-yl)-1-methyl-1H-pyrazol-5-yl)-4-methyl-1-naphthonitrile hydrochloride

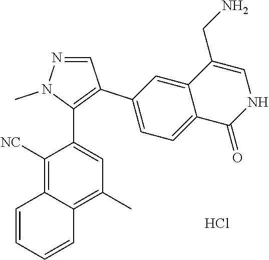 [SMILES: Cc1cc(-c2c(-c3ccc4c(=O)[nH]cc(CN)c4c3)cnn2C)c(C#N)c2ccccc12 ; Cl]

### Step 1: 2-iodo-4-methyl-1-naphthonitrile

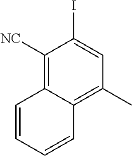

To a solution of 4-methyl-1-naphthonitrile (9.0 g, 53.8 mmol) in DCE (54 mL) was added NIS (14.5 g, 64.6 mmol), TsOH (4.63 g, 26.9 mmol) and Pd(OAc)₂ (1.21 g, 5.38 mmol). The mixture was stirred at 70° C. for 12 h. The reaction mixture was cooled to room temperature and concentrated under vacuum. The residue was purified by silica gel column chromatography (PE:EtOAc=1/0 to 0/1) to give the title compound (3.50 g) as an impure mixture. LC-MS (M+H)⁺=294.0.

### Step 2: 4-methyl-2-(1-methyl-1H-pyrazol-5-yl)-1-naphthonitrile

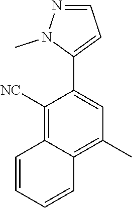

To a solution of 2-iodo-4-methyl-1-naphthonitrile (3.50 g, directly from step 1) and 1-methyl-5-(4,4,5,5-tetramethyl-1,3,2-dioxaborolan-2-yl)-1H-pyrazole (2.48 g, 11.9 mmol) in dioxane (20 mL) and water (4 mL) was added Na₂CO₃ (2.53 g, 23.9 mmol) and Pd(dtbpf)Cl₂ (778 mg, 1.19 mmol). The mixture was stirred at 80° C. for 12 h. The reaction mixture was cooled to room temperature, diluted with water (20 mL) and extracted with EtOAc (20 mL×2). The combined organic layer was concentrated under vacuum and the residue was purified by silica column chromatography (PE:EtOAc=100:1 to 0:1) to give the title compound (80 mg, 0.6% over 2 steps). LC-MS (M+H)⁺=248.0.

### Step 3: 2-(4-iodo-1-methyl-1H-pyrazol-5-yl)-4-methyl-1-naphthonitrile

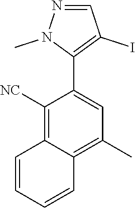 [SMILES: Cc1cc(-c2c(I)cnn2C)c(C#N)c2ccccc12]

To a solution of 4-methyl-2-(1-methyl-1H-pyrazol-5-yl)-1-naphthonitrile (80 mg, 0.32 mmol) in HOAc (1 mL) was added NIS (87 mg, 0.39 mmol). The mixture was stirred at 20° C. for 12 h. The mixture was concentrated under reduced pressure and the residue was purified by silica gel column chromatography to give the title compound (80 mg, 66%). LC-MS (M+H)⁺=374.1.

### Step 4: tert-butyl ((6-(5-(1-cyano-4-methylnaphthalen-2-yl)-1-methyl-1H-pyrazol-4-yl)-1-oxo-1,2-dihydroisoquinolin-4-yl)methyl)carbamate

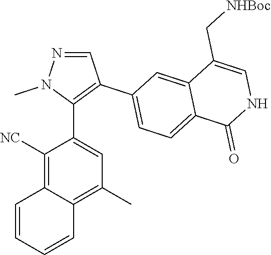 [SMILES: Cc1cc(-c2c(-c3ccc4c(=O)[nH]cc(CNC(=O)OC(C)(C)C)c4c3)cnn2C)c(C#N)c2ccccc12]

To a solution of 2-(4-iodo-1-methyl-1H-pyrazol-5-yl)-4-methyl-1-naphthonitrile (60 mg, 0.16 mmol) and tert-butyl ((1-oxo-6-(4,4,5,5-tetramethyl-1,3,2-dioxaborolan-2-yl)-1,2-dihydroisoquinolin-4-yl)methyl)carbamate (32 mg, 0.08 mmol) in dioxane (2 mL) and water (0.4 mL) was added Na₂CO₃ (43 mg, 0.40 mmol) and Pd(dppf)Cl₂ (12 mg, 0.016 mmol). The mixture was stirred at 80° C. for 3 h, cooled to room temperature and diluted with water (2 mL). The mixture was extracted with EtOAc (2 mL×2) and the combined organic layer was concentrated under reduced pressure. The residue was purified by silica gel chromatography to give the title compound (10 mg, 12%). LC-MS (M+H)⁺=520.0.

### Step 5: 2-(4-(4-(aminomethyl)-1-oxo-1,2-dihydroisoquinolin-6-yl)-1-methyl-1H-pyrazol-5-yl)-4-methyl-1-naphthonitrile hydrochloride

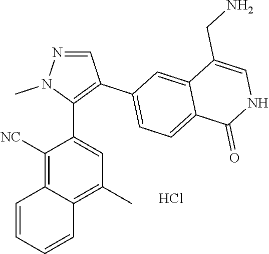 [SMILES: Cc1cc(-c2c(-c3ccc4c(=O)[nH]cc(CN)c4c3)cnn2C)c(C#N)c2ccccc12 ; Cl]

To a solution of tert-butyl ((6-(5-(1-cyano-4-methylnaphthalen-2-yl)-1-methyl-1H-pyrazol-4-yl)-1-oxo-1,2-dihydroisoquinolin-4-yl)methyl)carbamate (10 mg, 0.019 mmol) in MeOH (2 mL) was added HCl/MeOH (4 M, 0.005 mL). The mixture was stirred at 20° C. for 2 h, then concentrated under reduced pressure to give Example 4 (4 mg, 54%). ¹H NMR (400 MHZ, CD₃OD) δ 8.37-8.31 (m, 1H), 8.24-8.17 (m, 2H), 8.04 (d, J=8.4 Hz, 1H), 7.91 (s, 1H), 7.88-7.82 (m, 2H), 7.62 (s, 1H), 7.38 (s, 1H), 7.04 (dd, J=8.8, 1.2 Hz, 1H), 4.28-4.18 (m, 2H), 3.85 (s, 3H), 2.88 (s, 3H). LC-MS (M−NH₂)⁺=403.0.

### Example 5: 2-(4-(4-(aminomethyl)-1-oxo-1,2-dihydroisoquinolin-6-yl)-1-methyl-1H-pyrazol-5-yl)-4-chloro-1-naphthonitrile hydrochloride

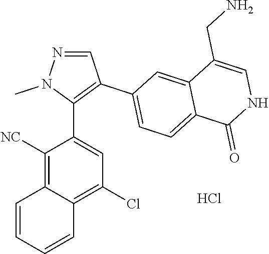 [SMILES: Cn1ncc(-c2ccc3c(=O)[nH]cc(CN)c3c2)c1-c1cc(Cl)c2ccccc2c1C#N ; Cl]

### Step 1: 4-chloro-2-iodo-1-naphthonitrile

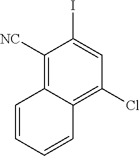 [SMILES: N#Cc1c(I)cc(Cl)c2ccccc12]

To a solution of 4-chloro-1-naphthonitrile (4.0 g, 21.3 mmol) in DCE (24 mL) was added NIS (5.28 g, 23.5 mmol), TsOH (1.84 g, 10.7 mmol) and Pd(OAc)₂ (479 mg, 2.13 mmol). The mixture was stirred at 70° C. for 12 h, cooled to room temperature and concentrated under vacuum. The residue was purified by prep-TLC to give the title compound (2.0 g, 30%).

### Step 2: 4-chloro-2-(1-methyl-1H-pyrazol-5-yl)-1-naphthonitrile

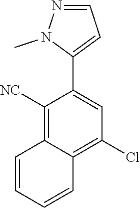 [SMILES: Cn1nccc1-c1cc(Cl)c2ccccc2c1C#N]

To a solution of 4-chloro-2-iodo-1-naphthonitrile (0.50 g, 1.59 mmol) and 1-methyl-5-(4,4,5,5-tetramethyl-1,3,2-dioxaborolan-2-yl)-1H-pyrazole (332 mg, 1.59 mmol) in dioxane (5 mL) and water (1 mL) was added NaHCO₃ (268 mg, 3.19 mmol) and Pd(dtbpf)Cl₂ (104 mg, 0.16 mmol). The mixture was stirred at 80° C. for 12 h. The mixture was cooled to room temperature, diluted with water (5 mL) and extracted with EtOAc (5 mL×2). The combined organic layer was concentrated under vacuum. The residue was purified by prep-TLC to give the title compound (50 mg, 12%). LC-MS (M+H)⁺=268.0.

### Step 3: 4-chloro-2-(4-iodo-1-methyl-1H-pyrazol-5-yl)-1-naphthonitrile

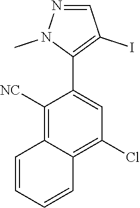 [SMILES: Cn1ncc(I)c1-c1cc(Cl)c2ccccc2c1C#N]

To a solution of 4-chloro-2-(1-methyl-1H-pyrazol-5-yl)-1-naphthonitrile (0.30 g, 1.12 mmol) in HOAc (2 mL) was added NIS (302 mg, 1.34 mmol). The mixture was stirred at 20° C. for 12 h then concentrated under vacuum. The residue was purified by silica gel chromatography to give the title compound (0.2 g, 45%). LC-MS (M+H)⁺=393.9.

### Step 4: tert-butyl ((6-(5-(4-chloro-1-cyanonaphthalen-2-yl)-1-methyl-1H-pyrazol-4-yl)-1-oxo-1,2-dihydroisoquinolin-4-yl)methyl)carbamate

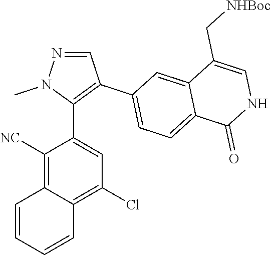 [SMILES: Cn1ncc(-c2ccc3c(=O)[nH]cc(CNC(=O)OC(C)(C)C)c3c2)c1-c1cc(Cl)c2ccccc2c1C#N]

To a solution of 4-chloro-2-(4-iodo-1-methyl-1H-pyrazol-5-yl)-1-naphthonitrile (50 mg, 0.13 mmol) and tert-butyl ((1-oxo-6-(4,4,5,5-tetramethyl-1,3,2-dioxaborolan-2-yl)-1,2-dihydroisoquinolin-4-yl)methyl)carbamate (25 mg, 0.063 mmol) in dioxane (1 mL) and water (0.2 mL) was added Na₂CO₃ (34 mg, 0.32 mmol) and Pd(dppf)Cl₂ (9 mg, 0.013 mmol). The mixture was stirred at 80° C. for 3 h. The reaction mixture was cooled to room temperature, diluted with water (2 mL) and extracted with EtOAc (2 mL×2). The combined organic layer was concentrated under vacuum. The residue was purified by silica gel chromatography to give the title compound (12 mg, 18%). LC-MS (M+H)⁺=540.2.

### Step 5: 2-(4-(4-(aminomethyl)-1-oxo-1,2-dihydroisoquinolin-6-yl)-1-methyl-1H-pyrazol-5-yl)-4-chloro-1-naphthonitrile hydrochloride

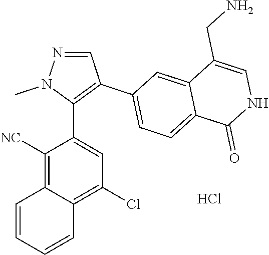 [SMILES: Cn1ncc(-c2ccc3c(=O)[nH]cc(CN)c3c2)c1-c1cc(Cl)c2ccccc2c1C#N ; Cl]

To a solution of tert-butyl ((6-(5-(4-chloro-1-cyanonaphthalen-2-yl)-1-methyl-1H-pyrazol-4-yl)-1-oxo-1,2-dihydroisoquinolin-4-yl)methyl)carbamate (10 mg, 0.019 mmol) in MeOH (2 mL) was added HCl in MeOH (4.0 M, 2 mL). The mixture was stirred at 20° C. for 3 h. The reaction mixture was concentrated under vacuum to give Example 5 (6 mg, 72%). ¹H NMR (400 MHZ, CD₃OD) δ 8.56-8.49 (m, 1H), 8.32-8.23 (m, 1H), 8.19 (s, 1H), 8.08 (d, J=8.4, 1H), 7.98-7.88 (m, 4H), 7.38 (s, 1H), 7.05 (d, J=8.4, 1H), 4.30-4.21 (m, 2H), 3.88 (s, 3H). LC-MS (M−NH₂)⁺=423.0.

### Example 6: 2-(4-(4-(aminomethyl)-1-oxo-1,2-dihydroisoquinolin-6-yl)-1-methyl-1H-pyrazol-5-yl)-6-cyclopropoxybenzonitrile hydrochloride

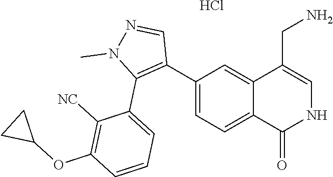 [SMILES: Cl ; Cn1ncc(-c2ccc3c(=O)[nH]cc(CN)c3c2)c1-c1cccc(OC2CC2)c1C#N]

### Step 1: 2-bromo-6-cyclopropoxybenzonitrile

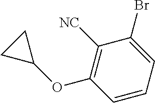 [SMILES: N#Cc1c(Br)cccc1OC1CC1]

To a solution of 2-bromo-6-fluorobenzonitrile (2.0 g, 10 mmol) and cyclopropanol (697 mg, 12 mmol) in DMF (50 mL) was added Cs₂CO₃ (8.15 g, 25 mmol) at 0° C. The mixture was warmed to room temperature and stirred for 4 h. The mixture was poured into water (150 mL), and successively extracted with EtOAc (200 mL×2). The combined organic layer was concentrated under vacuum to give the title compound (1.5 g, 63% yield).

### Step 2: 2-cyclopropoxy-6-(1-methyl-1H-pyrazol-5-yl)benzonitrile

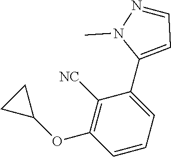 [SMILES: Cn1nccc1-c1cccc(OC2CC2)c1C#N]

To a solution of 2-bromo-6-cyclopropoxybenzonitrile (1.0 g, 4.20 mmol) and 1-methyl-5-(4,4,5,5-tetramethyl-1,3,2-dioxaborolan-2-yl)-1H-pyrazole (1.1 g, 5.04 mmol) in dioxane (10 mL) and water (2 mL) was added NaHCO₃ (0.88 g, 10.5 mmol) and Pd(dtbpf)Cl₂ (0.27 g, 0.42 mmol). The mixture was stirred at 80° C. for 16 h under nitrogen, cooled to room temperature and filtered. The filtrate was concentrated under vacuum and the residue was purified by silica gel column chromatography (PE:EtOAc=100:1 to 2:1) to give the title compound (0.75 g, 71%). LC-MS (M+H)⁺=240.3.

### Step 3: 2-cyclopropoxy-6-(4-iodo-1-methyl-1H-pyrazol-5-yl)benzonitrile

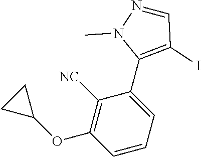 [SMILES: Cn1ncc(I)c1-c1cccc(OC2CC2)c1C#N]

To a solution of 2-cyclopropoxy-6-(1-methyl-1H-pyrazol-5-yl)benzonitrile (0.70 g, 2.93 mmol) in AcOH (4.2 mL) was added NIS (790 mg, 3.51 mmol). The mixture was stirred at 75° C. for 12 h, cooled to room temperature, poured to water (10 mL). The suspension was stirred for 1 h at room temperature and filtered, washed with water (10 mL), dried under reduced pressure to give the title compound (0.50 g, 45%). LC-MS (M+H)⁺=366.0.

### Step 4: tert-butyl ((6-(5-(2-cyano-3-cyclopropoxyphenyl)-1-methyl-1H-pyrazol-4-yl)-1-oxo-1,2-dihydroisoquinolin-4-yl)methyl)carbamate

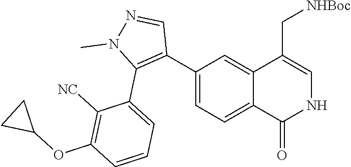 [SMILES: Cn1ncc(-c2ccc3c(=O)[nH]cc(CNC(=O)OC(C)(C)C)c3c2)c1-c1cccc(OC2CC2)c1C#N]

To a solution of 2-cyclopropoxy-6-(4-iodo-1-methyl-1H-pyrazol-5-yl)benzonitrile (100 mg, 0.27 mmol) and tert-butyl ((1-oxo-6-(4,4,5,5-tetramethyl-1,3,2-dioxaborolan-2-yl)-1,2-dihydroisoquinolin-4-yl)methyl)carbamate (55 mg, 0.14 mmol) in dioxane (1 mL) and water (0.2 mL) was added Na₂CO₃ (45 mg, 0.43 mmol) and Pd(dppf)Cl₂ (20 mg, 0.027 mmol). The mixture was stirred at 85° C. for 16 h, cooled to room temperature and filtered. The filtrate was concentrated under vacuum and the residue was purified by prep-TLC to give the title compound (20 mg, 14%). LC-MS (M+H)⁺=512.0.

### Step 5: 2-(4-(4-(aminomethyl)-1-oxo-1,2-dihydroisoquinolin-6-yl)-1-methyl-1H-pyrazol-5-yl)-6-cyclopropoxybenzonitrile hydrochloride

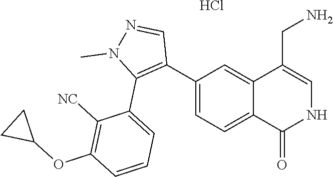 [SMILES: Cl ; Cn1ncc(-c2ccc3c(=O)[nH]cc(CN)c3c2)c1-c1cccc(OC2CC2)c1C#N]

To a solution of tert-butyl ((6-(5-(2-cyano-3-cyclopropoxyphenyl)-1-methyl-1H-pyrazol-4-yl)-1-oxo-1,2-dihydroisoquinolin-4-yl)methyl)carbamate (20 mg, 0.039 mmol) in MeOH (0.1 mL) was added HCl in MeOH (4.0 M, 0.05 mL). The mixture was stirred at 20° C. for 4 h then concentrated under vacuum. The residue was purified by prep-HPLC with water (HCl)/MeCN system to give Example 6 (11 mg, 66%). ¹H NMR (400 MHZ, CD₃OD) δ 8.18-8.11 (m, 2H), 7.88-7.82 (m, 2H), 7.75-7.67 (m, 1H), 7.40 (s, 1H), 7.59-7.53 (m, 1H), 7.14-7.07 (m, 1H), 4.32-4.20 (m, 2H), 4.10-4.02 (m, 1H), 3.79 (s, 3H). LC-MS (M−NH₂)⁺=395.0.

### Example 7: 2-(4-(4-(aminomethyl)-1-oxo-1,2-dihydroisoquinolin-6-yl)-1-methyl-1H-pyrazol-5-yl)-6-cyclopropoxy-4-(trifluoromethyl)benzonitrile hydrochloride

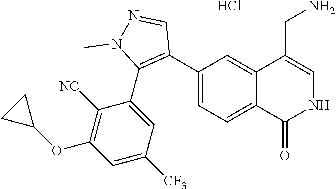 [SMILES: Cn1ncc(-c2ccc3c(=O)[nH]cc(CN)c3c2)c1-c1cc(C(F)(F)F)cc(OC2CC2)c1C#N ; Cl]

### Step 1: 2-bromo-6-fluoro-4-(trifluoromethyl) aniline

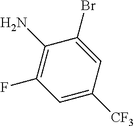 [SMILES: Nc1c(F)cc(C(F)(F)F)cc1Br]

To a solution of 2-fluoro-4-(trifluoromethyl) aniline (20.0 g, 112 mmol) in MeCN (30 mL) was added NBS (21.9 g, 123 mmol) at 15° C. The mixture was warmed to 85° C. and stirred for 3 h. The residue was partitioned between EtOAc (100 mL) and water (50 mL). The organic layer was separated, dried over Na₂SO₄, filtered and concentrated under reduced pressure to give the title compound (20.0 g, 69%).

### Step 2: 2-bromo-6-fluoro-4-(trifluoromethyl)benzonitrile

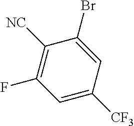 [SMILES: N#Cc1c(F)cc(C(F)(F)F)cc1Br]

To a solution of 2-bromo-6-fluoro-4-(trifluoromethyl) aniline (20.0 g, 77.5 mmol) in DCM (100 mL) was added nitronium tetrafluoroborate (10.0 g, 85.3 mmol) at 0° C., then the mixture was stirred at 0° C. for 1 h. KCN (15.1 g, 233 mmol) was added into the mixture, followed by CuSO₄ (24.74 g, 155 mmol) in water (100 mL). The mixture was warmed to room temperature and stirred for 12 h. The mixture was carefully concentrated under reduced pressure and the residue was partitioned between EtOAc (100 mL) and water (50 mL). The organic layer was separated, dried over Na₂SO₄, filtered and concentrated under reduced pressure. The residue was purified by silica gel column chromatography (PE:EtOAc=1:0 to 0:1) to give the title compound (3.0 g, 14%).

### Step 3: 2-bromo-6-cyclopropoxy-4-(trifluoromethyl)benzonitrile

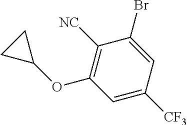 [SMILES: N#Cc1c(Br)cc(C(F)(F)F)cc1OC1CC1]

To a solution of 2-bromo-6-fluoro-4-(trifluoromethyl)benzonitrile (3.0 g, 11.2 mmol) in DMF (15 mL) was added Cs₂CO₃ (9.12 g, 28.0 mmol) and cyclopropanol (650 mg, 11.2 mmol) at 15° C., the mixture was stirred for 2 h. The mixture was partitioned between ethyl acetate (30 mL) and water (50 mL). The organic layer was separated, dried over Na₂SO₄, filtered and concentrated under reduced pressure. The residue was purified by silica gel column chromatography (PE:EtOAc=1:0 to 0:1) to give the title compound (1.0 g, 29%).

### Step 4: 2-cyclopropoxy-6-(1-methyl-1H-pyrazol-5-yl)-4-(trifluoromethyl)benzonitrile

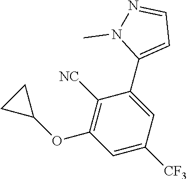 [SMILES: Cn1nccc1-c1cc(C(F)(F)F)cc(OC2CC2)c1C#N]

To a solution of 2-bromo-6-cyclopropoxy-4-(trifluoromethyl)benzonitrile (1.0 g, 3.27 mmol) in dioxane (1 mL) and water (0.1 mL) was added NaHCO₃ (823 mg, 9.80 mmol), 1-methyl-5-(4,4,5,5-tetramethyl-1,3,2-dioxaborolan-2-yl)-1H-pyrazole (680 mg, 3.27 mmol) and Pd(dtbpf)Cl₂ (106 mg, 0.16 mmol). The mixture was stirred at 80° C. for 12 h, cooled to room temperature, partitioned between EtOAc (30 mL) and water (50 mL). The organic layer was separated, dried over Na₂SO₄, filtered and concentrated under reduced pressure. The crude was purified by silica gel column chromatography (PE:EtOAc=1:0 to 0:1) to give the title compound (500 mg, 50%). ¹H NMR (400 MHZ, DMSO-d6) δ 7.86 (s, 1H), 7.66 (s, 1H), 7.58 (s, 1H), 6.58 (s, 1H), 4.32-4.27 (m, 1H), 3.78 (s, 3H), 1.00-0.88 (m, 2H), 0.87-0.80 (m, 2H).

### Step 5: 2-cyclopropoxy-6-(4-iodo-1-methyl-1H-pyrazol-5-yl)-4-(trifluoromethyl)benzonitrile

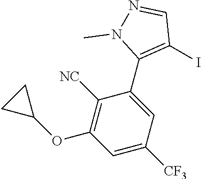 [SMILES: Cn1ncc(I)c1-c1cc(C(F)(F)F)cc(OC2CC2)c1C#N]

To a solution of 2-cyclopropoxy-6-(1-methyl-1H-pyrazol-5-yl)-4-(trifluoromethyl)benzonitrile (400 mg, 1.30 mmol) in AcOH (2 mL) was added NIS (586 mg, 2.60 mmol) at 15° C. The mixture was stirred for 12 h then concentrated under reduced pressure. The residue was partitioned between EtOAc (5 mL) and water (5 mL). The organic layer was separated, dried over Na₂SO₄ and concentrated under reduced pressure. The crude was triturated with MTBE (2 mL) for 30 min, filtered and dried under vacuum to give the title compound (100 mg, 18%). ¹H NMR (400 MHZ, DMSO-d6) δ 7.93 (s, 1H), 7.72 (s, 1H), 7.66 (s, 1H), 4.40-4.28 (m, 1H), 3.76 (s, 3H), 1.01-0.81 (m, 4H). LC-MS (M+H)⁺=434.0.

### Step 6: tert-butyl ((6-(5-(2-cyano-3-cyclopropoxy-5-(trifluoromethyl)phenyl)-1-methyl-1H-pyrazol-4-yl)-1-oxo-1,2-dihydroisoquinolin-4-yl)methyl)carbamate

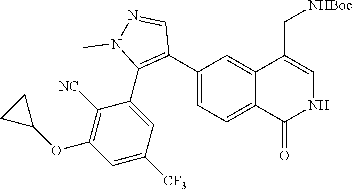 [SMILES: Cn1ncc(-c2ccc3c(=O)[nH]cc(CNC(=O)OC(C)(C)C)c3c2)c1-c1cc(C(F)(F)F)cc(OC2CC2)c1C#N]

To a solution of 2-cyclopropoxy-6-(4-iodo-1-methyl-1H-pyrazol-5-yl)-4-(trifluoromethyl)benzonitrile (90 mg, 0.21 mmol) in dioxane (1 mL) and water (0.2 mL) was added tert-butyl ((1-oxo-6-(4,4,5,5-tetramethyl-1,3,2-dioxaborolan-2-yl)-1,2-dihydroisoquinolin-4-yl)methyl)carbamate (42 mg, 0.10 mmol), Pd(dppf)Cl₂ (15 mg, 0.021 mmol) and NaHCO₃ (52 mg, 0.62 mmol) under N₂. The mixture was stirred at 80° C. for 3 h, cooled to room temperature and concentrated under reduced pressure. The residue was purified with prep-TLC to give the title compound (20 mg, 17%). LC-MS (M+H)⁺=580.2.

### Step 7: 2-(4-(4-(aminomethyl)-1-oxo-1,2-dihydroisoquinolin-6-yl)-1-methyl-1H-pyrazol-5-yl)-6-cyclopropoxy-4-(trifluoromethyl)benzonitrile hydrochloride

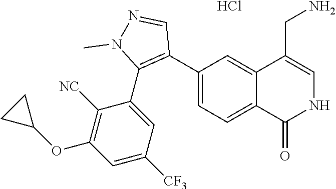 [SMILES: Cn1ncc(-c2ccc3c(=O)[nH]cc(CN)c3c2)c1-c1cc(C(F)(F)F)cc(OC2CC2)c1C#N ; Cl]

To a solution of tert-butyl ((6-(5-(2-cyano-3-cyclopropoxy-5-(trifluoromethyl)phenyl)-1-methyl-1H-pyrazol-4-yl)-1-oxo-1,2-dihydroisoquinolin-4-yl)methyl)carbamate (20 mg, 0.035 mmol) in MeOH (1 mL) was added HCl in MeOH (4.0 M, 0.01 mL) at 15° C., the mixture was stirred at 15° C. for 2 and concentrated under reduced pressure. The residue was purified by prep-HPLC with water (HCl)/MeCN system to give Example 7 (2 mg, 10%). ¹H NMR (400 MHZ, CD₃OD) δ 8.24-8.17 (m, 2H), 7.92-7.88 (m, 2H), 7.60 (s, 1H), 7.42 (s, 1H), 7.03 (dd, J=8.4, 1.2 Hz, 1H), 4.34-4.27 (m, 2H), 4.23-4.15 (m, 1H), 3.82 (s, 3H), 1.02-0.94 (m, 2H), 0.92-0.83 (m, 2H). LC-MS (M−NH₂)⁺=463.0.

### PRMT5 Biochemical Assay

The assay measures the methylation activity of purified human PRMT5/MEP50 enzyme toward histone H4-R3. Compounds disclosed herein were tested for inhibition of PRMT5/MEP50 using PRMT5 TR-FRET Assay Kit (BPS Bioscience) which contains a highly specific antibody that recognizes methylated substrates.

The assay was carried out in 384-well low volume black plates in a reaction mixture containing 10 nM PRMT5/MEP50 complex, biotinylated histone H₄ peptide, 3 μM S-adenosylmethionine and 0-10 μM compound in buffer containing 50 mM Tris-HCl buffer (pH 8.5), 0.005% BSA, 1 mM TCEP and 0.002% Tween-20. The PRMT5/MEP50 enzyme was incubated with compounds disclosed herein and biotinylated histone H₄ peptide for 20 minutes at room temperature. The reaction was initiated by addition of S-adenosylmethionine. After reacting at room temperature for 120 minutes, the detection solution containing Eu-labeled antibody and dye-labeled acceptor in detection buffer was added to the reaction mixture. Plates were sealed and incubated at room temperature for 60 minutes, and the TR-FRET signals (excitation 337 nm, emission 665/620 nm) were recorded on a PHERAstar FSX plate reader (BMG Labtech). The inhibition percentage of PRMT5/MEP50 activity in presence of increasing concentrations of compounds was calculated based on the ratio of fluorescence at 665 nm to that at 620 nm. The IC₅₀ value for each compound was derived from fitting the dose-response % inhibition data to the four-parameter logistic model by Dotmatics. And the compounds were tested in the presence and absence of MTA to evaluate whether the compounds display MTA-cooperative activity.

| | Biochemical IC₅₀ with | Biochemical IC₅₀ |
|---|---|---|
| Example | 800 nM MTA (nM) | without MTA (nM) |
| 1 | 3.2 | 29 |
| 2 | 3.3 | 77 |
| 3 | 7.6 | 68 |
| 4 | 2.7 | 29 |
| 5 | 2.7 | 27 |
| 6 | 3.5 | 87 |
| 7 | 6.2 | 165 |

What is claimed is:

1. A compound of Formula (I): 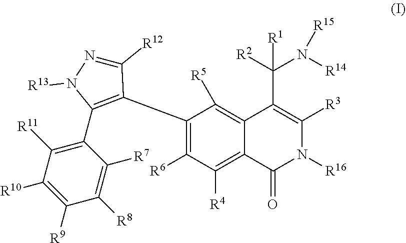
or a N-oxide thereof, or a pharmaceutically acceptable salt thereof, or a stereoisomer thereof, or a deuterated analog thereof, wherein:
R¹, R², R³, R⁴, R⁵, R⁶, R⁷, R⁸, R¹¹ and R¹² are each independently selected from hydrogen, halogen, —C₁₋₈alkyl, —C₃-C₈cycloalkyl, —CN, —OR^{1a}, —NR^{1a}R^{1b}, —COR^{1a}, —CO₂R^{1a}, —CONR^{1a}R^{1b} or —NR^{1a}COR^{1b}, wherein each of —C₁₋₈alkyl and —C₃-C₈cycloalkyl is optionally substituted with at least one substituent selected from halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl;
each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl;
each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁹ and R¹⁰ are each independently selected from hydrogen, halogen, —C₁₋₈alkyl, C₃-C₈cycloalkyl, —CN, —OR^{9a}, —NR^{9a}R^{9b} or —NR^{9a}COR^{9b}, wherein each of —C₁₋₈alkyl and C₃-C₈cycloalkyl is optionally substituted with at least one substituent R^{9d}; or
R⁹ and R¹⁰ together with the carbon atoms to which they are attached, form a 5-6 membered saturated or partially or completely unsaturated (preferably completely unsaturated, i.e., aromatic) ring, said ring comprising 0-3 heteroatoms independently selected from nitrogen, oxygen or sulfur; said ring is optionally substituted with at least one substituent R⁹;
R^{9e}, at each occurrence, is independently hydrogen, halogen, —C₁₋₈alkyl, —C₂₋₈alkenyl, —C₂₋₈alkynyl, —C₁₋₈alkoxy, —C₃-C₈cycloalkyl, oxo, 3- to 8-membered heterocyclyl, C₆-C₁₂aryl, 5- to 12-membered heteroaryl, —CN, —SO₂R^{9a}, —SO₂NR^{9a}R^{9b}, —COR^{9a}, —CO₂R⁹³, —CONR^{9a}R^{9b}, —NR^{9a}R^{9b}, —NR^{9a}COR^{9b}, —NR^{9a}CO₂R^{9b} or —NR^{9a}SO₂R^{9b}, wherein each of —C₁₋₈alkyl, —C₂₋₈alkenyl, —C₂₋₈alkynyl, —C₁₋₈alkoxy, C₃-C₈cycloalkyl, 3- to 8-membered heterocyclyl, C₆-C₁₂aryl or 5- to 12-membered heteroaryl is optionally substituted with at least one substituent R^{9d};
R^{9a} and R^{9b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R^{9f};
R^{9d} and R^{9f}, at each occurrence, are each independently halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R¹³, R¹⁴, R¹⁵ and R¹⁶ are each independently selected from hydrogen, —C₁₋₈alkyl, —C₃-C₈cycloalkyl or —C₆-C₁₂aryl, wherein each of —C₁₋₈alkyl, —C₃-C₈cycloalkyl and —C₆-C₁₂aryl is optionally substituted with at least one substituent selected from hydrogen, halogen, —C₁₋₈alkyl, —C₂₋₈alkenyl, —C₂₋₈alkynyl, —NR^{13a}R^{13b}, —OR^{13a}, oxo, —C₃-C₈cycloalkyl, 3- to 8-membered heterocyclyl, —C₆-C₁₂aryl, 5- to 12-membered heteroaryl, or —CN;
R^{13a} and R^{13b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, wherein each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{13c};
R^{13c} is independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl or —CN, wherein each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl is optionally substituted with at least one hydrogen, halogen, hydroxy, —C₁₋₈alkyl, —C₁₋₈alkoxy, —CN, —NH₂ or oxo.

2. The compound of claim 1, wherein the compound is selected from formula (IIa) or (IIb) 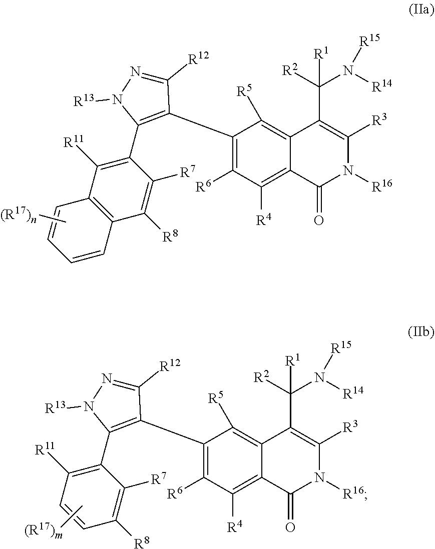
wherein
R¹, R², R³, R⁴, R⁵, R⁶, R⁷, R⁸, R¹¹, R¹², R¹³, R¹⁴, R¹⁵ and R¹⁶ are each as defined as claim 1; and at each of its occurrences, R¹⁷ is independently selected from hydrogen, halogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —SO₂R^{17a}, —SO₂NR^{17a}R^{17b}, —COR^{17a}, —CO₂R^{17a}, —CONR^{17a}R^{17b}, —OR^{17a}, —NR^{17a}R^{17b}, —NR^{17a}COR^{17b}, —NR^{17a}CO₂R^{17b}, or —NR^{17a}SO₂R^{17b}, wherein each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{17c}, —SO₂R^{17c}, —SO₂NR^{17c}R^{17d}, —COR^{17b}, —CO₂R^{17c}, —CONR^{17c}R^{17d}, —NR^{17c}R^{17d}, —NR^{17c}OR^{17d}, —NR^{17c}CO₂R^{17d}, or —NR^{17c}SO₂R^{17d}, R^{17a} and R^{17b} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl;
each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{17c} and R^{17d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl;
each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
n is 0, 1, 2, 3 or 4;
m is 0, 1 or 2.

3. The compound of claim 1 or claim 2, wherein the compound is selected from formula (IIIa) or (IIIb) 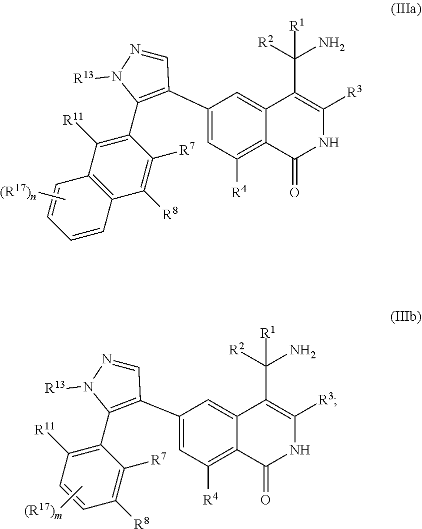
wherein
R¹, R², R³, R⁴, R⁵, R⁶, R⁷, R⁸, R¹¹, R¹², R¹³, R¹⁴, R¹⁵ and R¹⁶ are each as defined as claim 1; and R¹⁷, n and m are each defined as claim 2.

4. The compound of any one of the preceding claims, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b}, or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl and cycloheptyl, cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR¹⁴, —NR^{1c}CO₂R^{1d}, or —NR^{1c}SO₂R^{1d};
R^{1a}, R^{1b}, R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent selected from halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

5. The compound of any one of the preceding claims, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R¹³, —CONR^{1a}R^{1b}, —OR^{1a}, —NR^{1a}R^{1b}, or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

6. The compound of any one of the preceding claims, wherein R¹, R², R³, R⁴, R⁵, R⁶ and R¹² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl or cyclooctyl; preferably, R¹, R², R³, R⁴, R⁵, R⁶ and R¹² are each independently selected from hydrogen, methyl, ethyl, propyl, butyl, pentyl, hexyl, heptyl, octyl; more preferably, R¹, R², R³, R⁴, R⁵, R⁶ and R¹² are each independently selected from hydrogen.

7. The compound of any one of the preceding claims, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b}, or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

8. The compound of any one of the preceding claims, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1a}, —NR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

9. The compound of any one of the preceding claims, wherein R⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl or cyclooctyl; preferably, R⁷ is each independently selected from hydrogen, —F, —Cl, —Br, —I, methyl, ethyl, propyl, butyl, pentyl, hexyl, heptyl, octyl; more preferably, R⁷ is independently selected from hydrogen, —F, —Cl, —Br or —I.

10. The compound of any one of the preceding claims, wherein R⁸ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b}, —OR^{1d}, —NR^{1a}R^{1b} or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

11. The compound of any one of the preceding claims, wherein R⁸ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR¹⁴R¹⁵, —OR^{1a}, —NR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

12. The compound of any one of the preceding claims, wherein R⁸ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl or cyclooctyl;
preferably, R⁸ is each independently selected from hydrogen, —F, —Cl, —Br, —I, —CH₂F, —CHF₂, —CF₃, —C₂F₅, methyl, ethyl, propyl, butyl, pentyl, hexyl, heptyl, octyl; more preferably, R⁸ is independently selected from hydrogen, —CH₂F, —CHF₂, —CF₃, —C₂F₅, —F, —Cl, —Br or —I.

13. The compound of any one of the preceding claims, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{9a}, —NR^{9a}R^{9b} or —NR^{9a}COR^{9b}, wherein each of methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with at least one substituent R^{9d};
R^{9a} and R^{9b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9f};
R^{9d} and R^{9f}, at each occurrence, are each independently —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

14. The compound of any one of the preceding claims, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy, each of said methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

15. The compound of any one of the preceding claims, wherein R⁹ and R¹⁰ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, heptyloxy, octyloxy, cyclopropoxy, cyclobutoxy, cyclopentoxy, cyclohexoxy, cyclohepthoxy, cyclooctoxy.

16. The compound of anyone of claims 1-12, wherein R⁹ and R¹⁰ together with the carbon atoms to which they are attached, form a 5 or 6 membered saturated or partially or completely unsaturated (preferably completely unsaturated, i.e., aromatic) ring, said ring comprising 0, 1, 2 or 3 heteroatoms independently selected from nitrogen, oxygen or sulfur; said ring is optionally substituted with at least one substituent R^{9e};
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN, —SO₂R^{9a}, —SO₂NR^{9a}R^{9b}, —COR^{9a}, —CO₂R^{9a}, —CONR^{9a}R^{9b}, —NR^{9a}R^{9b}, —NR^{9a}COR^{9b}, —NR^{9a}CO₂R^{9b} or —NR^{9a}SO₂R^{9b}, wherein each of methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl or 5- to 12-membered heteroaryl is optionally substituted with at least one substituent R^{9a};
R^{9a} and R^{9b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9f};
R^{9d} and R^{9f}, at each occurrence, are each independently —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

17. The compound of anyone of claim 1-12 or 16, wherein R⁹ and R¹⁰ together with the carbon atoms to which they are attached, form a 5 or 6 membered aromatic ring, said ring comprising 0, 1, 2 or 3 heteroatoms independently selected from nitrogen, oxygen or sulfur; said ring is optionally substituted with at least one substituent R^{9e};
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN, wherein each of methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl or 5- to 12-membered heteroaryl is optionally substituted with at least one substituent —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

18. The compound of anyone of claim 1-12 or 16-17, wherein R⁹ and R¹⁰ together with the carbon atoms to which they are attached, form a phenyl ring; said ring is optionally substituted with at least one substituent R^{9e};
R^{9e}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, oxo, 3- to 8-membered heterocyclyl, phenyl, 5- to 12-membered heteroaryl, —CN.

19. The compound of any one of the preceding claims, wherein R¹¹ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b} or —NR^{1a}COR^{1b}, wherein each of said methyl, ethyl, propyl, butyl, pentyl, hexyl, heptyl, octyl, cyclopropyl, cyclobutyl, cyclopentyl, cyclohexyl, cycloheptyl and cyclooctyl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}OR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one halogen, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

20. The compound of any one of the preceding claims, wherein R¹¹ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —COR^{1a}, —CO₂R^{1a}, —CONR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

21. The compound of any one of the preceding claims, wherein R¹¹ is independently selected from hydrogen, —F, —Cl, —Br, —I, —CH₂F, —CHF₂, —CF₃, —C₂F₅, methyl, ethyl, propyl, butyl, pentyl, hexyl, heptyl, octyl, cyclopropyl, cyclobutyl, cyclopentyl, cyclohexyl, cycloheptyl, cyclooctyl or —CN; preferably, R¹¹ is each independently selected from hydrogen, —F, —Cl, —Br, —I, —CH₂F, —CHF₂, —CF₃, —C₂F₅, methyl, ethyl, propyl, butyl, pentyl, hexyl, heptyl, octyl or —CN; more preferably, R¹¹ is —CN.

22. The compound of any one of the preceding claims, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl, wherein each of methyl, ethyl, propyl, butyl, pentyl, hexyl, heptyl, octyl, cyclopropyl, cyclobutyl, cyclopentyl, cyclohexyl, cycloheptyl, cyclooctyl and phenyl is optionally substituted with at least one substituent elected from hydrogen, —F, —Cl, —Br, —I, methyl, ethyl, propyl, butyl, pentyl, hexyl, heptyl, octyl, —C₂₋₈alkenyl, —C₂₋₈alkynyl, —NR^{13a}R^{13b}, —OR^{13a}, oxo, cyclopropyl, cyclobutyl, cyclopentyl, cyclohexyl, cycloheptyl, cyclooctyl, 3- to 8-membered heterocyclyl, phenyl, 5- to 12-membered heteroaryl, or —CN;
R^{13a} and R^{13b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{13c};
R^{13c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN, wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one hydrogen, —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —CN, —NH₂ or oxo.

23. The compound of any one of the preceding claims, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl, wherein each of methyl, ethyl, propyl, butyl, pentyl, hexyl, heptyl, octyl, cyclopropyl, cyclobutyl, cyclopentyl, cyclohexyl, cycloheptyl, cyclooctyl and phenyl is optionally substituted with at least one substituent elected from hydrogen, —F, —Cl, —Br, —I, methyl, ethyl, propyl, butyl, pentyl, hexyl, heptyl, octyl, —C₂₋₈alkenyl, —C₂₋₈alkynyl, —NR^{13a}R^{13b}, —OR^{13a}, oxo, cyclopropyl, cyclobutyl, cyclopentyl, cyclohexyl, cycloheptyl, cyclooctyl, 3- to 8-membered heterocyclyl, phenyl, 5- to 12-membered heteroaryl, or —CN;
R^{13a} and R^{13b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

24. The compound of any one of the preceding claims, wherein R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or phenyl; preferably R¹³, R¹⁴, R¹⁵, R¹⁶ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl; more preferably R¹³, R¹⁴, R¹⁵, R¹⁶ are each independently selected from hydrogen, methyl, ethyl, propyl, butyl, pentyl, hexyl, heptyl, octyl.

25. The compound of any one of the preceding claims, wherein R¹³, R¹⁴, R¹⁵, R¹⁶ are each independently selected from hydrogen, methyl; preferably R¹³ is methyl and R¹⁴, R¹⁵ and R¹⁶ are each hydrogen.

26. The compound of any one of the preceding claims, wherein R¹⁷ is independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —SO₂R^{17a}, —SO₂NR^{17a}R^{17c}, —COR^{17a}, —CO₂R^{17a}, —CONR^{17a}R^{17c}, —OR^{17a}, —NR^{17a}R^{17b}, —NR^{17a}COR^{17b} NR^{17a}CO₂R^{17b}, or —NR^{17a}SO₂R^{17b}, wherein each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with —F, —Cl, —Br, —I, methoxy, ethoxy, propoxy, butoxy, pentoxy, hexoxy, heptyloxy, octyloxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{17c}, —SO₂R^{17c}, —SO₂NR^{17c}R^{17d}, —COR^{17c}, —CO₂R^{17c}, —CONR^{17c}R^{17d}, —NR^{17c}R^{17d}, —NR^{17c}OR^{17d}, —NR^{17c}CO₂R^{17d} or —NR¹⁷SO₂R^{17d};
R^{17a} and R^{17b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one-F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{17c} and R^{17d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one-F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

27. The compound of any one of the preceding claims, wherein R¹⁷ is independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —SO₂R^{17a}, —SO₂NR^{17a}R^{17c}, —COR^{17d}, —CO₂R^{17a}, —CONR^{17a}R^{17b}, —OR^{17a}, —NR^{17a}R^{17b}, —NR^{17a}COR^{17b}, —NR^{17a}CO₂R^{17b}, or —NR^{17a}SO₂R^{17b};
R^{17a} and R^{17b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one-F, —Cl, —Br, —I, —OH, methyl, ethyl, propyl, butyl, pentyl, hexyl, heptyl, octyl, methoxy, ethoxy, propoxy, butoxy, pentoxy, hexoxy, heptyloxy, octyloxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

28. The compound of any one of the preceding claims, wherein R¹⁷ is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, —CN, cyclopropoxy, cyclobutoxy, cyclopentoxy, cyclohexoxy, cyclohepthoxy, cyclooctoxy.

29. The compound of any one of the preceding claims, wherein the 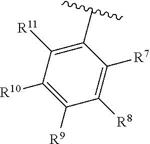
moiety is

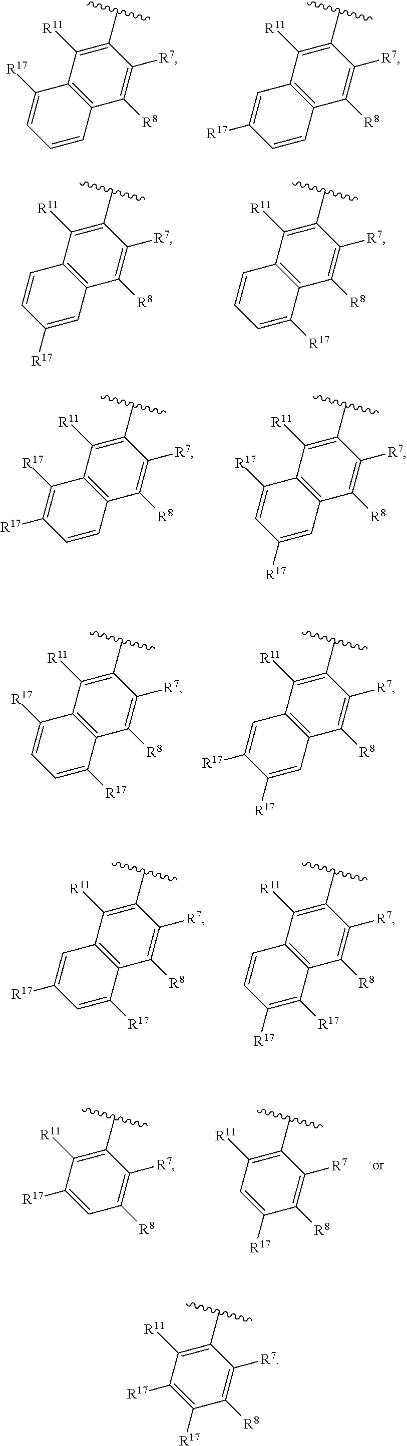

30. The compound of any one of the preceding claims, wherein the 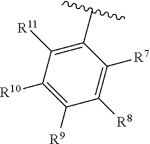
moiety is

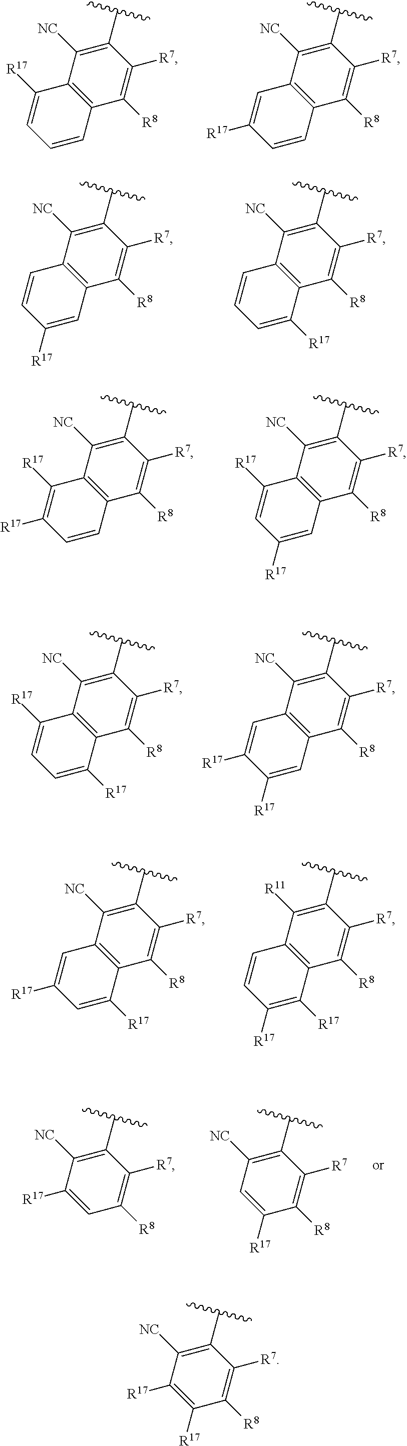

31. The compound of any one of the preceding claims, wherein the 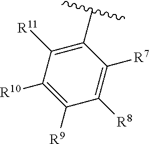
moiety is

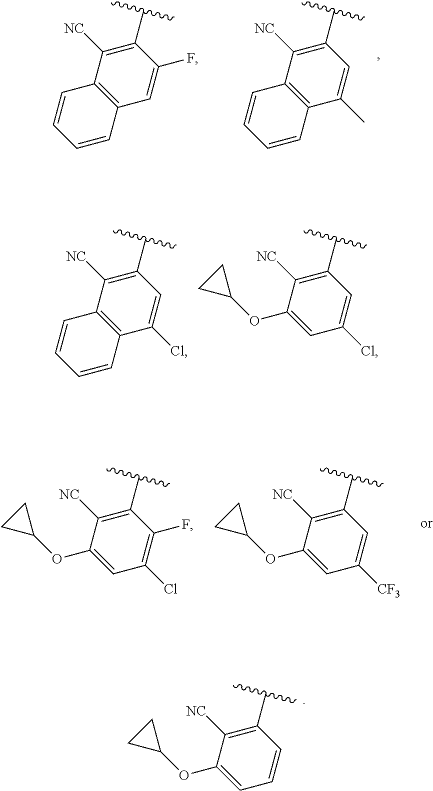

32. The compound of any one of the preceding claims, wherein the compound is selected from

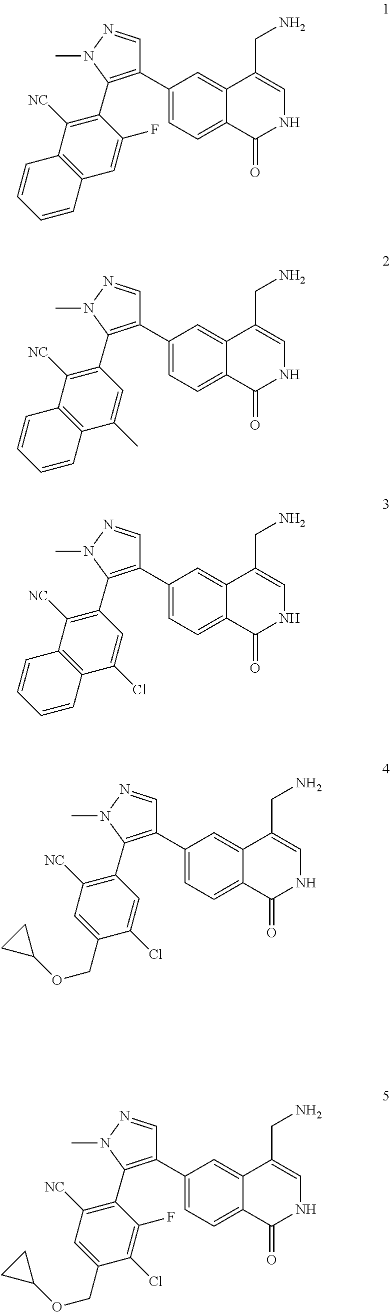 [SMILES: Cn1ncc(-c2ccc3c(=O)[nH]cc(CN)c3c2)c1-c1c(F)cc2ccccc2c1C#N ; Cc1cc(-c2c(-c3ccc4c(=O)[nH]cc(CN)c4c3)cnn2C)c(C#N)c2ccccc12 ; Cn1ncc(-c2ccc3c(=O)[nH]cc(CN)c3c2)c1-c1cc(Cl)c2ccccc2c1C#N ; Cn1ncc(-c2ccc3c(=O)[nH]cc(CN)c3c2)c1-c1cc(Cl)c(COC2CC2)cc1C#N ; Cn1ncc(-c2ccc3c(=O)[nH]cc(CN)c3c2)c1-c1c(C#N)cc(COC2CC2)c(Cl)c1F]

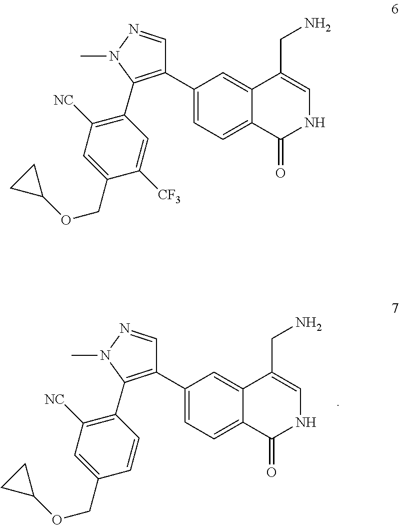 [SMILES: Cn1ncc(-c2ccc3c(=O)[nH]cc(CN)c3c2)c1-c1cc(C(F)(F)F)c(COC2CC2)cc1C#N ; Cn1ncc(-c2ccc3c(=O)[nH]cc(CN)c3c2)c1-c1ccc(COC2CC2)cc1C#N]

33. A pharmaceutical composition comprising a compound of any one of claims 1-32 or a pharmaceutically acceptable salt, stereoisomer, tautomer or prodrug thereof, together with a pharmaceutically acceptable excipient.

34. A method of decreasing PRMT5 activity by inhibition, which comprises administering to an individual the compound according to any one of claims 1-33, or a pharmaceutically acceptable salt thereof, including the compound of formula (I) or the specific compounds exemplified herein.

35. The method of claim 34, wherein the disease is selected from cancer.

36. Use of a compound of any one of claims 1-32 or a pharmaceutically acceptable salt, stereoisomer, tautomer or prodrug thereof in the preparation of a medicament for treating a disease that is modulated by PRMT5.

37. The use of claim 36, wherein the disease is cancer.

38. The use of claim 36, wherein the disease is MTAP-null solid tumor, including but not limited to lung cancer, bladder cancer, melanoma, pancreatic cancer, esophageal cancer, gastric adenocarcinoma, breast cancer, glioblastoma, etc.
